SEQUENCE LISTING (14 sequences)

SEQ ID NO: 1 - protein, 14 aa, Artificial Sequence
  Tryptic peptide containing potential ubiquitination sites of SUMO-2
VAGQDGSVVQFKIK

SEQ ID NO: 2 - protein, 9 aa, Artificial Sequence
  Tryptic peptides containing potential ubiquitination sites of SUMO-2
HTPLSKLMK

SEQ ID NO: 3 - protein, 14 aa, Artificial Sequence
  Tryptic peptides containing potential ubiquitination sites of SUMO-2
EGVKTENNDHINLK

SEQ ID NO: 4 - protein, 95 aa, Homo sapiens
MADEKPKEGVKTENNDHINLKVAGQDGSVVQFKIKRHTPLSKLMKAYCERQGLSMRQIRFRFDGQPINETDTPAQLEMEDEDTIDVFQQQTGGVY

SEQ ID NO: 5 - protein, 4 aa, Mus musculus
ITVI

SEQ ID NO: 6 - protein, 4 aa, Mus musculus
LTVI

SEQ ID NO: 7 - protein, 4 aa, Mus musculus
LQLV

SEQ ID NO: 8 - protein, 4 aa, Mus musculus
IIPL

SEQ ID NO: 9 - protein, 4 aa, Mus musculus
LWVI

SEQ ID NO: 10 - protein, 4 aa, Mus musculus
LIWV

SEQ ID NO: 11 - protein, 4 aa, mouse
LEVL

SEQ ID NO: 12 - protein, 295 aa, Homo sapiens
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 13 - protein, 854 aa, Mus musculus
MGSLTMKSQLQITVISAKLKENKKNWFGPSPYVEVTVDGQSKKTEKCNNTNSPKWKQPLTVIVTPTSKLCFRVWSHQTLKSDVLLGTAGLDIYETLKSNNMKLEEVVMTLQLVGDKEPTETMGDLSVCLDGLQVEAEVVTNGETSCSESTTQNDDGCRTRDDTRVSTNGSEDPEVAASGENKRANGNNSPSLSNGGFKPSRPPRPSRPPPPTPRRPASVNGSPSTNSDSDGSSTGSLPPTNTNVNTSTSEGATSGLIIPLTISGGSGPRPLNTVSQAPLPPGWEQRVDQHGRVYYVDHVEKRTTWDRPEPLPPGWERRVDNMGRIYYVDHFTRTTTWQRPTLESVRNYEQWQLQRSQLQGAMQQFNQRFIYGNQDLFATSQNKEFDPLGPLPPGWEKRTDSNGRVYFVNHNTRITQWEDPRSQGQLNEKPLPEGWEMRFTVDGIPYFVDHNRRATTYIDPRTGKSALDNGPQIAYVRDFKAKVQYFRFWCQQLAMPQHIKITVTRKTLFEDSFQQIMSFSPQDLRRRLWVIFPGEEGLDYGGVAREWFFLLSHEVLNPMYCLFEYAGKDNYCLQINPASYINPDHLKYFRFIGRFIAMALFHGKFIDTGFSLPFYKRILNKPVGLKDLESIDPEFYNSLIWVKENNIEECGLEMYFSVDKEILGEIKSHDLKPNGGNILVTEENKEEYIRMVAEWRLSRGVEEQTQAFFEGFNEILPQQYLQYFDAKELEVLLCGMQEIDLNDWQRHAIYRHYTRTSKQIMWFWQFVKEIDNEKRMRLLQFVTGTCRLPVGGFADLMGSNGPQKFCIEKVGKENWLPRSHTCFNRLDLPPYKSYEQLKEKLLFAIEETEGFGQE

SEQ ID NO: 14 - protein, 4 aa, Artificial Sequence
  Synthetic SUMO interaction motif
XXXX

(19) United States
(12) Patent Application Publication
(10) Pub. No.: US 2016/0015743 A1
(43) Pub. Date: Jan. 21, 2016

(54) METHODS FOR TREATING CANCER

(71) Applicant: RUSH UNIVERSITY MEDICAL CENTER, Chicago, IL (US)

(72) Inventor: Di Chen, Chicago, IL (US)

(73) Assignee: RUSH UNIVERSITY MEDICAL CENTER, CHICAGO, IL (US)

(21) Appl. No.: 14/774,247

(22) PCT Filed: Mar. 13, 2014
(86) PCT No.: PCT/US14/25459

§ 371 Date: Sep. 10, 2015

Related U.S. Application Data
(60) Provisional application No. 61/799,888, filed on Mar. 15, 2013.

(51) Int. Cl. A61K 33/36

(52) U.S. Cl. CPC A61K 33/36

(57) ABSTRACT

Disclosed herein are methods for treating cancer, such as a cyclin D1-overexpressing cancer, including administering to a subject in need of such treatment a composition comprising a therapeutically effective amount of an agent that mediates downregulation of cyclin D1 and/or increases sumoylation of cyclin D1.

### RELATED APPLICATIONS

The present patent application claims the benefit of the filing date of U.S. Provisional Patent Application No. 61/799,888, filed Mar. 15, 2013, the contents of which is hereby incorporated by reference.

### STATEMENT OF GOVERNMENT INTEREST

This invention was made with government support under contract number RO1 AR055915-01A2 awarded by the National Institutes of Health. The government has certain rights in the invention.

### TECHNICAL FIELD

The present invention relates to methods for treating cancer.

### BACKGROUND

Cyclin D1 is a critical cyclin protein regulating G1-S phase transition during normal cell cycle progression (1). FIG. 12 shows the amino acid sequence of this protein (SEQ ID NO.: 12.) Multiple regulatory mechanisms are involved to maintain steady-state cyclin D1 protein levels under control in every second (2-3). Loss of control of cyclin D1 results in several disease outcomes. Overexpression of cyclin D1 was found in various types of cancers, such as breast, lung, prostate and bladder cancers (4-8). CCND1 functions as a driver gene which contributes to tumorigenesis.

### SUMMARY

Provided herein is a method for treating cancer. The method may comprise administering to a subject in need of such treatment a composition comprising a therapeutically effective amount of an agent that mediates downregulation of cyclin D1. The cancer may be selected from the group consisting of breast cancer, lung cancer, prostate cancer, and bladder cancer. The agent may mediate downregulation of cyclin D1 by increasing sumoylation of cyclin D1. The agent may be arsenic trioxide. The agent may upregulate activity of at least one of an E3 ligase and a SUMO-conjugating enzyme. The E3 ligase may be Itch. The SUMO-conjugating enzyme may be Ubc9.

Also provided herein is a method for treating a cyclin D1-overexpressing cancer. The method may comprise administering to a subject in need of such treatment a composition comprising a therapeutically effective amount of an agent that increases sumoylation of cyclin D1. The cancer may be selected from the group consisting of breast cancer, lung cancer, prostate cancer, and bladder cancer. The agent may be arsenic trioxide.

Further provided herein is a method of identifying a subject for treatment with an agent that increases sumoylation of cyclin D1. The method may include obtaining a biological sample comprising at least one cancer cell expressing cyclin D1 from the subject. The method may also include identifying the subject as being suitable for treatment with the agent based on detecting at least one sumoylation site in cyclin D1, and identifying the subject as being unsuitable for treatment with the agent based on detecting no sumoylation site in cyclin D1. The agent may be arsenic trioxide. The at least one sumoylation site may be a lysine residue in an amino acid sequence of cyclin D1. The lysine residue may be at position 149 in the amino acid sequence of cyclin D1. The subject identified as being suitable for treatment may be administered a composition comprising a therapeutically effective amount of the agent.

### BRIEF DESCRIPTION OF THE DRAWINGS

FIG. 1 shows SUMOylation is involved in mediating cyclin D1 proteasomal degradation. Ubc9 (a), SUMO1 (b), SUMO2 (c), SUMO3 (d) together with SENP1 constructs was co-transfected with cyclin D1 into HEK293 cells in the absence or presence of proteasome inhibitor MG132 (10 μM, 6 h of incubation). Cyclin D1 protein levels were detected through western blotting. (e) siRNA specific for Ubc9 was transiently transfected in human colon cancer cell HCT116 cells. Endogenous cyclin D1 protein levels were detected through western blotting. (f) In vivo SUMOylation and ubiquitination assay. HA-cyclin D1 was co-transfected with Ubc9 and SENP1 expression plasmids into HEK293 cells in the presence of MG132 (10 μM, 6 h of incubation). 24 h after the transfection, the cell lysates were collected, and SUMOylated as well as ubiquitinated proteins were pulled down using a specific SUMO-binding affinity matrix (SUMO-Qapture-T matrix, Enzo Life Science) or a specific ubiquitin-binding affinity matrix (UbiQapture-Q, Enzo Life Science), and SUMOylated cyclin D1 or polyubiquitinated cyclin D1 was detected using the anti-cyclin D1 antibody.

FIG. 2 shows mass spectrometry detecting SUMO-dependent cyclin D1 ubiquitination in a selected reaction monitoring mode (SRM). (a) Flow chart of the experiment. Empty vector or HA-cyclin D1 construct was transiently transfected into HEK293 cells. The HA-cyclin D1 transfected cells were cultured in two different conditions: growth medium or serum-free medium. 48 h after the transfection (MG132 treatment, 10 μM, 6 h before harvest), immunoprecipitation of cyclin D1 was performed using anti-HA antibody. The purified HA-cyclin D1 was confirmed by comaasie blue staining and western blotting (b). Then the cyclin D1 protein was digested by trypsin into small peptides for mass spectrometry detection in a LC-SRM mode. Three standard peptides were synthesized to detect the ubiquitination of SUMO-2 on three different potential sites. (c) SRM to identify the Ubiquitination sites of SUMO-2 in cyclin D1 precipitates. Tryptic peptides containing potential ubiquitination sites of SUMO-2 (VAGQDGSVVQFKIK (SEQ ID NO:1), HTPLSKLMK (SEQ ID NO:2), EGVKTENNDHINLK (SEQ ID NO:3)) are synthesized with the modification of two glycines being covalently linked to the lysine (underlined) in the sequence through an iso-peptide bond. SRM analysis of Trypsin-digested cyclin D1 precipitates of each indicated control or transfected group was performed by the Agilent 6460 QqQ Mass Spectrometer connected with Agilent 1260 HPLC. Ubiquitination of SUMO-2 was identified on the lysine within the sequence EGVKTENNDHINLK (SEQ ID NO:3) from the cells transfected with cyclin D1 construct and cultured in growth medium.

FIG. 3 shows lysine 149 is the critical site for cyclin D1 SUMOylation. (a) Through analyzing cyclin D1 protein sequence using program SUMOsp2.0, a series of point mutations in cyclin D1 protein were generated using site-directed mutagenesis kit (promega). The wt or mutant cyclin D1 constructs were co-transfected with Ubc9 into HEK293 cells. 24 h later, cyclin D1 protein expressions were detected through western blotting. (WT, wild type) (b) In vitro SUMOylation assay. HA-tagged wt cyclin D1 or cyclin D1 (K149R) were transfected into HEK293 cells. 24 h later, cyclin D1 proteins were purified by immunoprecipitation assay using anti-HA antibody. Then the cyclin D1 proteins were incubated in the presence of SUMO activating enzyme E1, conjugating enzyme Ubc9, SUMO-2, and ATP for 1 h (30° C.) (Enzo Life Science). Then the SUMOylated cyclin D1 was detected using anti-SUMO-2 antibody through western blotting. (c) WT cyclin D1 or cyclin D1 (K149R) construct was co-transfected with Ubc9 into HEK293 cells in the absence or presence of MG132 (10 μM, 6 h of incubation). Cyclin D1 protein levels were detected by western blotting using anti-HA antibody.

FIG. 4 shows blockage of both SUMOylation and phosphorylation stabilizes cyclin D1 protein. (a) Protein decay assay. WT or mutant cyclin D1 (K149R, T286A and DM) construct was transfected into HEK293 cells. 24 h after the transfection, the protein synthesis was blocked by cycloheximide treatment (50 μg/ml) for 6 h. Cell lysates were harvested at different time points (0, 30, 60, 120, and 300 mins). Cyclin D1 protein levels were detected by western blotting. (b) WT or mutant cyclin D1 (K149R, T286A and DM) construct was co-transfected with Ubc9 (E2 enzyme during SUMOylation) or DDB2 (E3 ligase which mediates phosphorylated cyclin D1 degradation) into HEK293 cells. Cyclin D1 protein levels were detected by western blotting. (c) Luciferase assay detecting the activities of wt or mutant cyclin D1. WT or mutant cyclin D1 (K149R, T286A and DM) constructs were co-transfected with E2F-luc reporter construct into HEK293 cells. Luciferase assay were performed 48 h after the transfection. Data are presented as means±SD of three independent experiments (* p<0.05, compared with wt group).

FIG. 5 shows SUMOylation participates in regulating cyclin D1 protein level during normal cell cycle progression. HCT-116 cells were synchronized before G1 phase through serum starvation for over 16 h. Then the cells were cultured with growth culture medium for 12 h. The cells were harvested at different time points (0, 3, and 12 h). Flow cytometry was performed to make sure that most of the cells had entered into the S phase at the 12 h time point. Then phospho-cyclin D1 or SUMOylated cyclin D1 were detected by western blotting using anti-phospho-cyclin D1 antibody (c) or co-immunoprecipitation assay (b, as described in FIG. 1f). (d) Flow cytometry to detect the cell cycle progression rates among the WT and mutant cyclin D1 constructs. WT or mutant cyclin D1 (K149R, T286A and DM) constructs were stably transfected into HCT-116 cells. The cells were synchronized before G1 phase through serum starvation for over 16 h. Then the cell cycle progression was released by changing the culture medium into growth medium. The cells were harvested at different time points (0, 6, 12, and 24 h) and cell cycle was detected through flow cytometry. (e) Cell proliferation assay. Stable transfected with WT cyclin D1 or mutant cyclin D1 (K149R, T286A and K149R/T286A) HCT-116, osteosarcoma cells U2OS, and human prostate cancer cells PC-3 were stained with crystal violet 5 days after the cells were seeded. (f) Soft agar assay in which cells stably expressing WT cyclin D1, cyclin D1-DM or empty vector were seeded at a density of 2×10³ cells per 35-mm dish and cultured in 0.35% soft agar in DMEM plus 10% FBS at 37° C. for 10 days. Colonies were visualized by microscopy. Data were shown as with 7×/50× magnifications. (g) Cyclin D1-DM accelerates growth of HCT-116 cells allografts in nude mice. i) Human colon cancer cells HCT-116 stably expressing WT cyclin D1 or cyclin D1-DM were grafted into athymic nude mice with 0.5×10⁶ cells per injection. The changes in average tumor volumes are shown as a function of time in i. (n=8 per group; *p<0.05). Error bars show SD. ii) Tumors were isolated 20 days after the graft then tumor weights were measured. The data of mean tumor weight in DM-cyclin D1 group is significantly higher than WT cyclin D1, indicating that the tumor cells grow more rapidly than WT cyclin D1 (n=8 per group; *p<0.05).

FIG. 6 shows Itch, functions as an E3 ligase, mediates cyclin D1 proteasomal degradation in a SUMOylation dependent manner. (a) Cell lysates were extracted from different tissues of wt or Itch-KO mice. Endogenous cyclin D1 protein levels were detected using anti-cyclin D1 antibody by western blotting. (b) siRNA specific for Ubc9 or SUMO-2 were co-transfected with Itch expression construct into HCT-116 cells. Endogenous cyclin D1 protein levels were detected using anti-cyclin D1 antibody by western blotting. (c) HA-tagged wt or mutant cyclin D1 (K149R, T286A) construct was co-transfected with Itch into HEK293 cells in the absence or presence of MG132 (10 μM, 6 h of incubation). Cyclin D1 protein levels were detected using anti-HA antibody by western blotting. (d) In vivo ubiquitination assay. HA-tagged wt or mutant cyclin D1 (K149R, T286A) construct was co-transfected with Itch and SENP1 expression plasmids into HEK293 cells in the presence of MG132 (10 μM, 6 h of incubation). 24 h after transfection, the cell lysates were collected, the ubiquitylated cyclin D1 was detected as described in FIG. 1f. (e) Itch construct with mutation on single SIM (112, 530 and 730) or with triple mutations was co-transfected with HA-tagged WT cyclin D1 or cyclin D1 (K149R) into HEK293 cells. Cyclin D1 protein levels were detected using anti-HA antibody by western blotting. (f) co-immunoprecipitation assay. Itch construct with mutation on single SIM (112, 530 and 730) or with triple mutations was transiently transfected into HCT-116 cells in the presence of MG132 (10 μM, 6 h of incubation). 24 h after transfection, IP was performed using the anti-Myc antibody followed by Western blotting using the anti-cyclin D1 antibody (top panel). To further detect the interaction between Itch and cyclin D1, co-IP assay were also performed using anti-cyclin D1 antibody followed by Western blotting using the anti-Myc antibody (middle panel).

FIG. 7 shows Arsenic trioxide (As₂O₃) induces cyclin D1 proteasomal degradation in a SUMO-triggered manner. (a, b&d) In vivo SUMOylation and ubiquitination assay. As for (a), HCT-116 cells were treated with As₂O₃ for 16 h (2.5 μM). Cell lysates were harvested at different time points (0, 1, 4 and 16 h). As for (b), WT or mutant cyclin D1 (K149R, T286A, DM) construct was stably transfected into HCT-116 cells. Then the cells were treated with As₂O₃ for 1 h. As for (d), HCT-116 cells were treated with As2O3 for 16 h (2.5 μM) in the absence or presence of MG132 (10 μM, 6 h of incubation). The SUMOylated and ubiquitylated cyclin D1 was detected as described in FIG. 1f. Cyclin D1 protein levels were detected using anti-cyclin D1 antibody (a&d) or anti-HA-antibody (b) by western blotting. (c) WT or mutant cyclin D1 (K149R, T286A, DM) construct was stably transfected into HCT-116 cells. Then the cells were treated with As₂O₃ for 16 h. Cyclin D1 protein levels were detected using anti-HA-antibody by western blotting. (e) TUNEL staining. WT or mutant cyclin D1 (K149R, T286A) construct was stably transfected into HCT-116 cells. Then the cells were treated with As₂O₃ (2.5 μM) for 16 h. The apoptotic cells were detected using Promega's DEADEND Colorimetric TUNEL System. Yellow arrows are pointing at apoptotic cells. (f) Flow cytometry. WT or mutant cyclin D1 (K149R) construct was stably transfected into HCT-116 cells. Then the cells were treated with As₂O₃ for 16 h (2.5 μM). Cell cycle progression was detected by flow cytometry. (As, arsenic trioxide)

FIG. 8 shows proteasome system is involved in regulating SUMOylated cyclin D1 protein level. HA-tagged cyclin D1 were co-transfected with Flag-tagged SUMO2 or Ubc9 into HEK293 cells in the absence or presence of MG132 (10 μM, 6 h of incubation). 24 h later, the cell lysates were extracted for co-immunoprecipitation assay. IP was performed using the anti-HA antibody followed by Western blotting using the anti-Flag antibody (top panel). Cyclin D1 protein levels were detected by western blotting (bottom panel).

FIG. 9 shows Cyclin D1-DM is the most stable form among the wt and mutant cyclin D1 constructs. The protein decay assay was performed as described in FIG. 4a. HEK293 cells transfected with cyclin D1-DM were treated with cycloheximide (50 μg/ml) for a longer period (12 h) than that in FIG. 4a.

FIG. 10 shows flow cytometry to detect the cell cycle progression rates among the wt and mutant cyclin D1 constructs in PC-3 cells (a) or U2OS cells (b). The experiment was performed as described in FIG. 5d.

FIG. 11 shows silencing of Itch did not block cyclin D1 degradation induced by As₂O₃. siRNA specific for Itch was transfected into HCT-116 cells in the absence or presence of As₂O₃ (2.5 μM). 48 h later, the cyclin D1 protein levels were detected through western blotting.

### DETAILED DESCRIPTION

One aspect of the present invention generally relates to methods of treatment of cancer in a human or veterinary subject. In one embodiment, the cancer cells overexpress cyclin D1. The cancer may be, for example, a breast cancer, a lung cancer, a prostate cancer, or a bladder cancer. The method may include administering to a subject in need of such treatment a composition including a therapeutically effective amount of an agent that mediates downregulation of cyclin D1. In certain embodiments, the agent mediates downregulation of cyclin D1 by increasing sumoylation of cyclin D1. In one preferred embodiment, the agent is arsenic trioxide.

The inventors have discovered that cyclin D1 is sumoylated at lysine 149 by a SUMO-conjugating enzyme such as Ubc9. The inventors have also shown that sumoylated cyclin D1 is ubiquinated by an E3 ligase such as Itch, thereby mediating downregulation of cyclin D1 via proteasome degradation of cyclin D1. The protein sequence of murine Itch is shown is FIG. 12(b) (SEQ ID NO.: 13) The inventors have further shown that mutation of lysine 149 of cyclin D1 prevented sumoylation, and thus degradation of cyclin D1. Mutation of lysine 149 of cyclin D1 promoted tumor growth.

Another aspect of the present invention provides methods of identifying a subject for treatment with an agent that increases sumoylation of cyclin D1. The agent may be arsenic trioxide. The method may include obtaining a biological sample including at least one cancer cell expressing cyclin D1 from the subject. The subject may be identified as being suitable for treatment with the agent if at least one sumoylation site is detected in cyclin D1. The at least one sumoylation site may be lysine 149 in cyclin D1. Such a suitable subject may be administered a composition including a therapeutically effective amount of the agent. Alternatively, the subject may be identified as being unsuitable for treatment with the agent if no sumoylation site is detected in cyclin D1.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. METHODS OF TREATING A CANCER

Provided herein is a method of treating a cancer. The cancer may be, for example, breast cancer, lung cancer, prostate cancer, or bladder cancer. The cancer may overexpress cyclin D1. Cyclin D1 regulates the cell cycle, namely the G1 to S phase transition. Overexpression of cyclin D1 and/or loss of cyclin D1 degradation may lead to tumorgenesis, neoplastic growth, or cancer by promoting or driving the cell cycle.

Cyclin D1, encoded by the CCND1 gene, is a critical cyclin protein for G1/S phase transition during normal cell cycle progression. Multiple regulatory mechanisms are involved to maintain cyclin D1 levels under proper control. Loss of control of cyclin D1 can result in diseases in humans. Abnormal up-regulation of cyclin D1 is found in various types of cancers, such as breast cancer, lung cancer, prostate cancer, bladder cancer and osteosarcoma.

The present disclosure demonstrates a novel modification mechanism of cyclin D1-SUMOylation and provides a method of treating a cancer. SUMOylation is a form of post-translational modification that regulates the cellular localization of modified proteins. Small ubiquitin-like modifiers (SUMOs) are ubiquitin-like polypeptides that become covalently conjugated to cellular proteins in a manner similar to ubiquitylation. In vertebrates, three SUMO isoforms are expressed. SUMO-1 shares 43% identity with SUMO-2 and SUMO-3, whereas the latter two are closely related (sharing 97% identity).

The method may include administering to a subject suffering from cancer a composition comprising an agent. The agent may downregulate or decrease cyclin D1 activity.

a. Cyclin D1-Overexpressing Cancers

The cyclin D1-overexpressing cancers may include cancers that have increased activity of cyclin D1. Such cyclin D1-overexpressing cancers may include, but are not limited to, breast cancer, lung cancer, prostate cancer, and bladder cancer.

Increased activity of cyclin D1 may result from increased levels of cyclin D1 protein, increased levels of cyclin D1 mRNA transcript, amplification of a cyclin D1 gene (i.e., change in cyclin D1 gene copy number), altered levels of cyclin D1 phosphorylation, altered levels of cyclin D1 ubiquination, altered levels of cyclin D1 sumoylation, and/or altered levels of cyclin D1 degradation. Cyclin D1 may be a substrate of a SUMO-conjugating enzyme, for example, Ubc9. Cyclin D1 may be sumoylated at lysine 149. Sumoylated cyclin D1 may be a substrate for an E3 ligase, for example, Itch. An E3 ligase may ubiquinate cyclin D1. Ubiquinated cyclin D1 may be a substrate for degradation by the proteasome.

Inability to sumoylate cyclin D1 may lead to overexpression of cyclin D1. Inability to sumoylate cyclin D1, and thus degrade cyclin D1, may promote progression through the cell cycle. Promoting progression through the cell cycle may promote tumorgenesis, neoplasm formation, neoplastic growth, and/or cancer. Inability to sumoylate cyclin D1 may occur by mutating or changing the codon that encodes for lysine 149 of cyclin D1 to encode for an amino acid residue other than lysine. Alternatively, deletion of the codon encoding for lysine 149 of cyclin D1 may result in inability to sumoylate cyclin D1.

Cyclin D1 may also be phosphorylated. Phosphorylation of cyclin D1 may lead to ubiquination of cyclin D1, and therefore, degradation of cyclin D1 by the proteasome. Phosphorylation of cyclin D1 may occur independently of sumoylation of cyclin D1. Alternatively, sumoylation of cyclin D1 may occur independently of phosphorylation of cyclin D1. Inability to phosphorylate and sumoylate cyclin D1 may lead to overexpression of cyclin D1. Inability to phosphorylate and sumoylate cyclin D1 may promote progression through the cell cycle. Promoting progression through the cell cycle may promote tumorgenesis, neoplasm formation, neoplastic growth, and/or cancer.

b. Agent

The agent may mediate downregulation of cyclin D1. Downregulation of cyclin D1 may occur by promoting or increasing sumoylation of cyclin D1, thereby causing ubiquination and degradation of cyclin D1. The agent may activate or upregulate a SUMO-conjugating enzyme such as Ubc9.

(1) Arsenic Trioxide

The agent mediating downregulation of cyclin D1 may be arsenic trioxide. Arsenic trioxide may increase or promote sumoylation of cyclin D1. Such sumoylation of cyclin D1 may lead to or increase ubiquination of cyclin D1 and subsequent degradation of cyclin D1 via the proteasome. Arsenic trioxide may increase sumoylation of unphosphorylated and/or phosphorylated cyclin D1. Arsenic trioxide may increase sumoylation of cyclin D1 independent of phosphorylation of cyclin D1. Sumoylation of cyclin D1 mediated by arsenic trioxide may occur at lysine 149 of the cyclin D1 protein.

Arsenic trioxide may mediate degradation of cyclin D1 in the absence of the E3 ligase, Itch. Arsenic trioxide may mediate degradation of cyclin D1 via any number of E3 ligases or ubiquitin conjugating enzymes. Arsenic trioxide may accelerate or increase the rate of apoptosis of cells. Arsenic trioxide may induce G1 arrest of the cell cycle. Such apoptosis and/or arrest of the cell cycle may be mediated by the sumoylation of cyclin D1, and subsequent ubiquination and degradation of cyclin D1. Sumoylation of cyclin D1 that leads to G1 arrest of the cell cycle and/or apoptosis may occur at lysine 149 of the cyclin D1 protein.

c. Pharmaceutical Compositions

The agent may be incorporated into pharmaceutical compositions suitable for administration to a subject (such as a patient, which may be a human or non-human).

The pharmaceutical compositions may include a “therapeutically effective amount” or a “prophylactically effective amount” of the agent. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of the agent are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

For example, a therapeutically effective amount of arsenic trioxide may be between about 0.5 mg/kg and 12 mg/kg, between about 1 mg/kg and 10 mg/kg, about 3 mg/kg and 7 mg/kg or between 4 mg/kg and 6 mg/kg.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

d. Modes of Administration

Methods for treating cancer may include any number of modes of administering the agent or pharmaceutical compositions of the agent. Modes of administration may include tablets, pills, dragees, hard and soft gel capsules, granules, pellets, aqueous, lipid, oily or other solutions, emulsions such as oil-in-water emulsions, liposomes, aqueous or oily suspensions, syrups, elixirs, solid emulsions, solid dispersions or dispersible powders. For the preparation of pharmaceutical compositions for oral administration, the agent may be admixed with commonly known and used adjuvants and excipients such as for example, gum arabic, talcum, starch, sugars (such as, e.g., mannitose, methyl cellulose, lactose), gelatin, surface-active agents, magnesium stearate, aqueous or non-aqueous solvents, paraffin derivatives, cross-linking agents, dispersants, emulsifiers, lubricants, conserving agents, flavoring agents (e.g., ethereal oils), solubility enhancers (e.g., benzyl benzoate or benzyl alcohol) or bioavailability enhancers (e.g. GELUCIRE). In the pharmaceutical composition, the agent may also be dispersed in a microparticle, e.g. a nanoparticulate, composition.

For parenteral administration, the agent or pharmaceutical compositions of the agent can be dissolved or suspended in a physiologically acceptable diluent, such as, e.g., water, buffer, oils with or without solubilizers, surface-active agents, dispersants or emulsifiers. As oils for example and without limitation, olive oil, peanut oil, cottonseed oil, soybean oil, castor oil and sesame oil may be used. More generally spoken, for parenteral administration the agent or pharmaceutical compositions of the agent can be in the form of an aqueous, lipid, oily or other kind of solution or suspension or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

### 3. METHODS OF IDENTIFICATION

Provided herein are methods of identifying a subject for treatment with the agent. The method may include obtaining a biological sample including at least one cell expressing cyclin D1 from the subject. The at least one cell expressing cyclin D1 may be a cancer cell.

The subject may be identified as being suitable for treatment with the agent if at least one sumoylation site is detected in cyclin D1. The at least one sumoylation site may be a lysine residue. The lysine residue may be lysine 149 in cyclin D1 protein. Such a suitable subject may be administered a composition including a therapeutically effective amount of the agent. Alternatively, the subject may be identified as being unsuitable for treatment with the agent if no sumoylation site is detected in cyclin D1.

The present invention has multiple aspects, illustrated by the following non-limiting examples.

### 4. EXAMPLES

### Example 1

### Materials and Methods for Examples 2-5

Western Blotting, Immunoprecipitation and Ubiquitylation Assay.

Western blotting and immunoprecipitation (IP) were performed as previously described (54). The interaction between endogenous Cyclin D1 and Itch was determined in HEK293 cells. Proteasome inhibitor MG132 (10 μM) (Sigma, St. Louis, Mo.) was added to the cell culture 6 hours before cells were harvested for immunoprecipitation assay. Blots were probed with the following antibodies: anti-human cyclin D1 mouse monoclonal (Cell Signaling), anti-phospho cyclin D1 (T286) rabbit polyclonal (Cell Signaling), anti-β-actin mouse monoclonal (Sigma), anti-HA mouse monoclonal (Roche), anti-myc mouse monoclonal (Sigma), anti-phospho-Rb (Ser780) (Cell Signaling).

In Vivo SUMOylation and Ubiquitylation Assay.

SUMOylated cyclin D1 or ubiquitylated cyclin D1 was detected by co-immunoprecipitation using anti-SUMO-2/3 antibody or anti-ubiquitin antibody conjugated beads (Enzo Life Science), followed by immunoblotting with anti-cyclin D1 antibody or anti-HA antibody for cyclin D1 detection.

Cell Cycle Analysis.

FACS analysis was performed as described in the research by Santra et al (55). For FACS analysis, HCT-116, U2OS or PC-3 cells were stably transfected with wt Cyclin D1 or mutant Cyclin D1 (K149R, T286A, DM). In some experiments, the cells were synchronized before the G1 phase through serum starvation for over 16 h or treated with As₂O₃ (2.5 μM) for 16 h. The cells were then stained with propidim iodide (50 μg/ml) at 37° C. for 1 h. FACS samples were analyzed with a FACSCANTO Flow Cytometry System (BD Biosciences). And the data were analyzed using FlowJo 7.6 software according to the manufacturer's instruction.

In Vitro SUMOylation Assay.

This experiment was performed using SUMOylation kit (Enzo Life Science). HA-tagged Cyclin D1 construct was transiently transfected into HEK293 cells. 48 h after the transfection, the cyclin D1 protein was purified using Pierce HA Tag IP/Co-IP Kit (Pierce). The purified cyclin D1 protein was incubated in the presence of ATP, SUMO-2, SUMO E1 and SUMO E2 for 1 h (30° C.). SUMOylated cyclin D1 were detected using anti-SUMO-2/3 antibody.

Allograft Mice Model.

This experiment was performed as described in the research by Kim et al. (52). HCT-116 cells stably transfected with wt cyclin D1 or cycin D1 (DM) were injected into two flank regions of athymic nude mice (Charles River Laboratories) with equal volumes of cells. Mice were weighed daily and watched for tumor formation. Once tumor appeared, tumor width and length were measured at different time points. Tumor volumes were calculated by considering the average value of width and length of tumor as the radius of a sphere and using the formula for the volume of sphere: V=4/3πr3. Tumor weights were also measured after the mice were sacrificed. Comparisons between wt and DM groups were done also using an unpaired-t test. Statistical significance was indicated by the P value (*p<0.05).

TUNEL Staining.

Cell apoptosis was detected using fluorescent in situ terminal deoxynucleotidyl transferase-mediated uridine 5′-triphosphate-biotin nick end labeling (TUNEL staining). Sections were first permeabilized in 0.1% Triton X-100 in phosphate-buffered saline (PBS) for 8 mins. TUNEL reaction mixture was obtained by adding terminal deoxynucleotidyl transferase to nucleotide mixture, as instructed by the manufacturer's manual (DEADEND Fluorometric TUNEL System, Promega). Sections were counterstained nuclei with 4′-6-Diamidino-2-phenylindole (DAPI).

Cell Proliferation Assay.

Anchorage-dependent cell proliferation was observed by crystal violet staining. Anchorage-independent cell proliferation was determined by a soft agar assay. Cells were seeded at a density of 2×10³ cells per 35-mm cell culture dish in 0.35% agar and cultured for 14 days at 37° C. under 5% CO2. Dishes were stained with 0.05% crystal violet. Colonies were counted in the entire dish, and the colony size was determined by a microcaliper.

LC-SRM and Data Analysis.

Validation of Cyclin D1 ubiquitination sites was performed as described in the research by Qing et al (17). Tryptic peptides representing each of the 3 potential ubiquitination sites were synthesized and analyzed through the Selected Reaction Monitoring (SRM) approach with the MS parameters as follows: drying gas: 12 L/min, 300° C.; fragmentor: 130 V; dwell time: 10 ms; capillary voltage: 4,000 V; resolution of Q1 and Q3: unit mass; collision energy: optimized for each peptide with the Agilent MassHunter Peptide Optimizer. SRM analysis was carried out in positive mode using a 6460 Triple Quadrupole Mass Spectrometer (Agilent Technologies) equipped with capillary flow (100 μL/min) electrospray ionization connected to an Agilent 1200 series capillary pump. The Skyline program preloaded with ubiquitylated Cyclin D1 peptide sequences was used to analyze the data (56).

Cell Culture and Transfection.

Human colon cancer HCT-116 cells, human osteosarcoma U205 cells and human embryonic kidney 293 (HEK293) cells were cultured in Dulbecco's modified Eagle's medium (DMEM) and human prostate cancer PC-3 cells were cultured in DMEM/F12 supplemented with 10% fetal calf serum at 37° C. under 5% CO2. HCT-116, U2OS, PC-3 cell lines expressing HA-Cyclin D1 or HA-Cyclin D1 (K149R, T286A, DM) were generated by transient transfection using Lipofectamine 2000 (Invitrogen, Carlsbad, Calif.). Then transfected colonies were selected in the presence of G418 (1000 μg/ml for HCT-116 cells; 500 μg/ml for U205 cells; 800 μg/ml for PC-3 cells). DNA plasmids were transiently transfected into cells in 6-cm culture dishes using Lipofectamine 2000. Empty vector was used to keep the total amount of transfected DNA plasmid constant in each group in all experiments. Flag-EGFP plasmid was co-transfected as an internal control to evaluate transfection efficiency. Western blotting and immunoprecipitation (IP) assays were performed 24 hours after transfection.

Plasmids and Site-Directed Mutagenesis.

Plasmids expressing HA-cyclin D1 and HA-cyclin D1(T286A) (57), Itch (58) were purchased from Addgene. Mutant cyclin D1 (K149R), cyclin D1 (DM, K149R/T286A) and loss of function mutants of Itch (L112A, V530A, V730A, L112A/V530A/V730A) were generated using site directed mutagenesis kit (Agilent, California, USA). All constructs were confirmed by sequencing.

In Vivo Protein Decay Assay.

Cells were seeded in 15-cm culture dishes, wt or mutant Cyclin D1 (K149R, T286A, DM) construct was transiently transfected, respectively, into HEK293 cells. 24 hrs after transfection, cells were trypsinized and split into five 10-cm dishes. 12 hrs after recovery, cells were cultured in regular medium with 80 μg/ml cycloheximide (Calbiochem, La Jolla, Calif.), for 0, 30, 60, 120, and 300 minutes before harvesting. Western blotting was performed to detect the decay of Cyclin D1 proteins.

Luciferase and Real Time PCR Assays.

The plasmids of reporter constructs were co-transfected with 3×E2F-luc reporter construct and cyclin D1 expression plasmid into HEK293 cells. 24 h after transfection, the cell lysates were then collected, and luciferase activity was measured using a Promega Dual Luciferase reporter assay kit.

Statistics.

Statistical comparison between two groups was performed using unpaired Student's t-test. p<0.05 was considered significant and is denoted in the figures.

### Example 2

### Cyclin D1 can be Degraded Through SUMO-Triggered Ubiquitin-Mediated Pathway

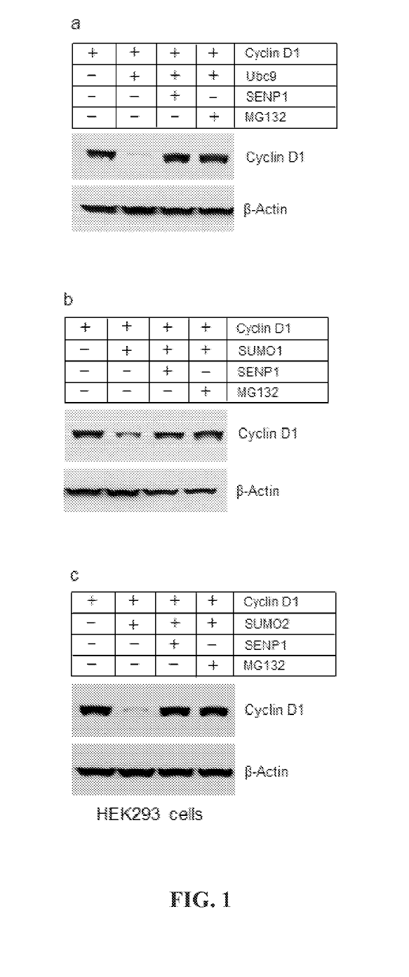

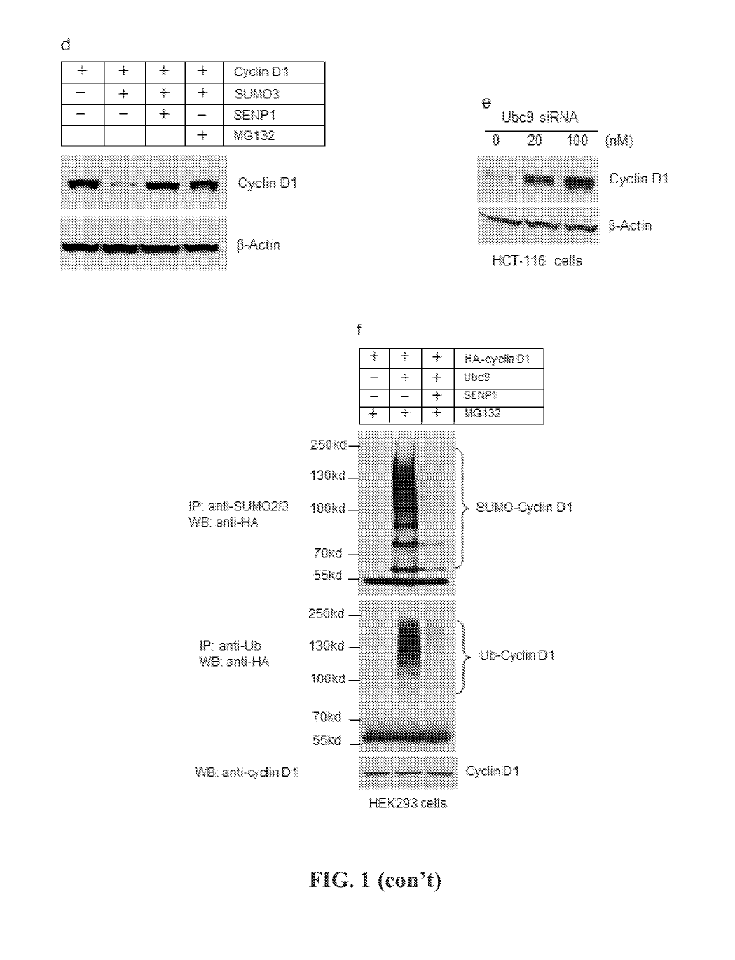

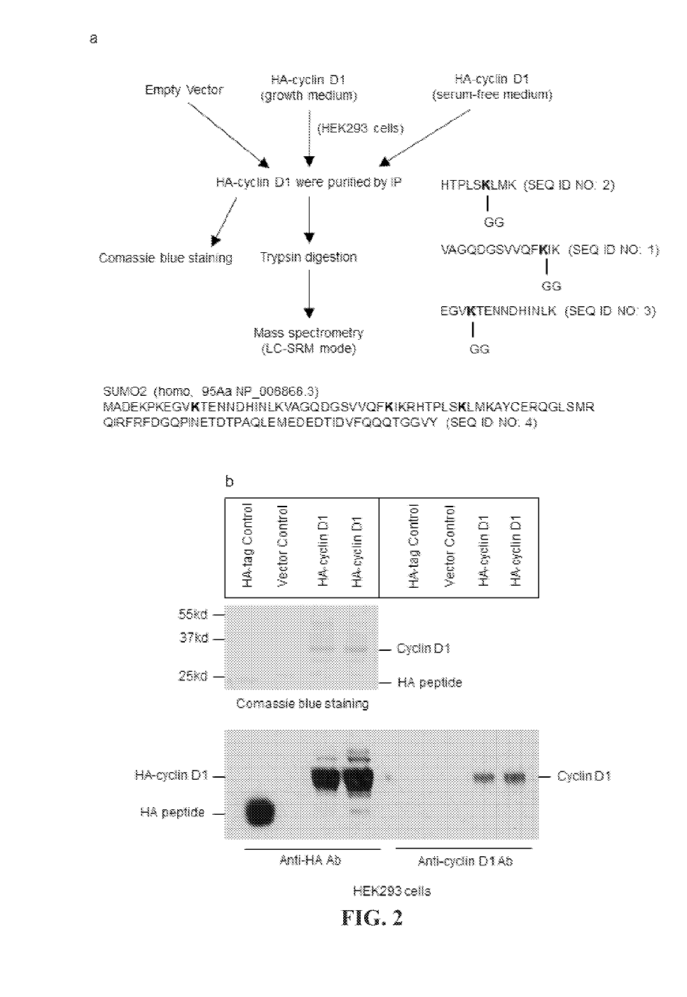

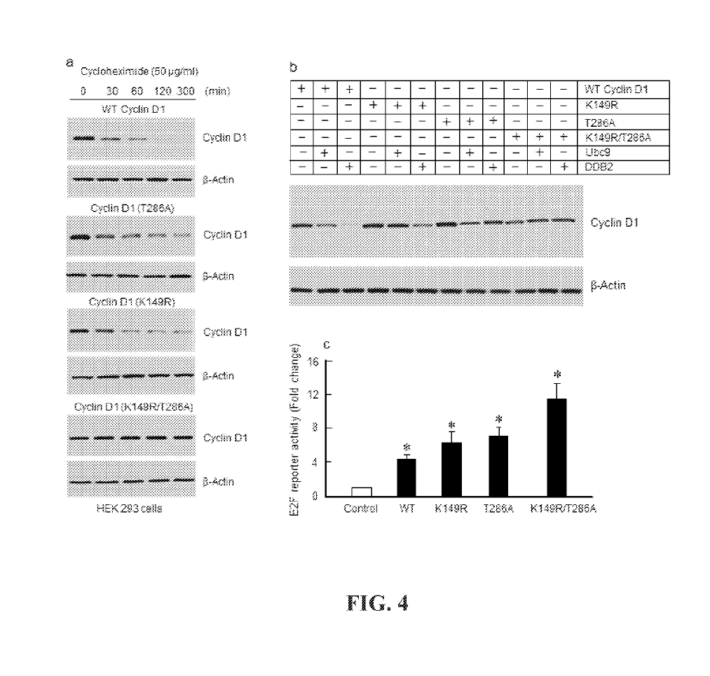

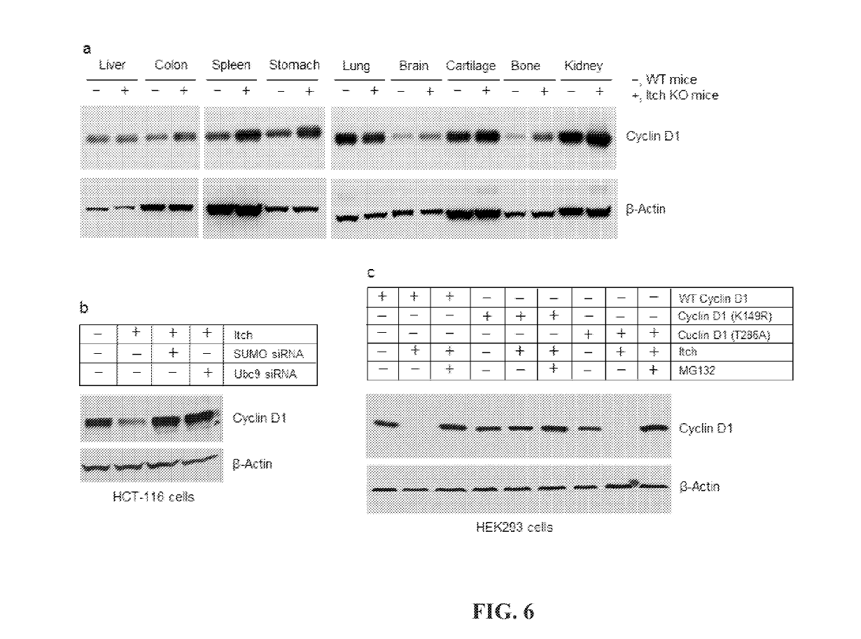

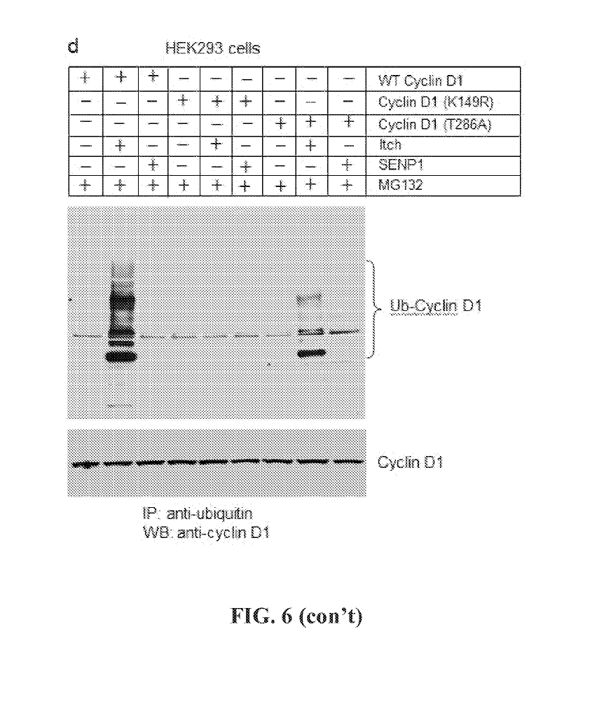

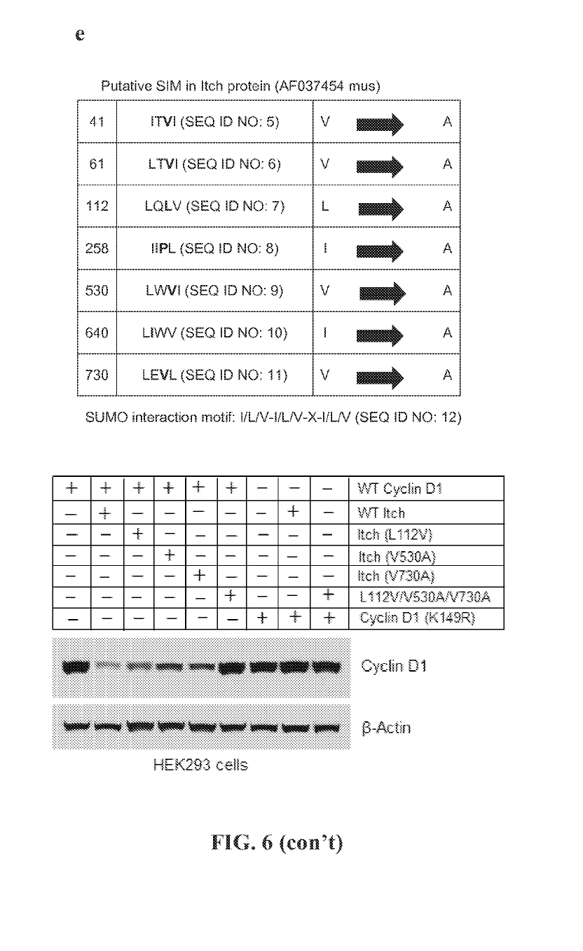

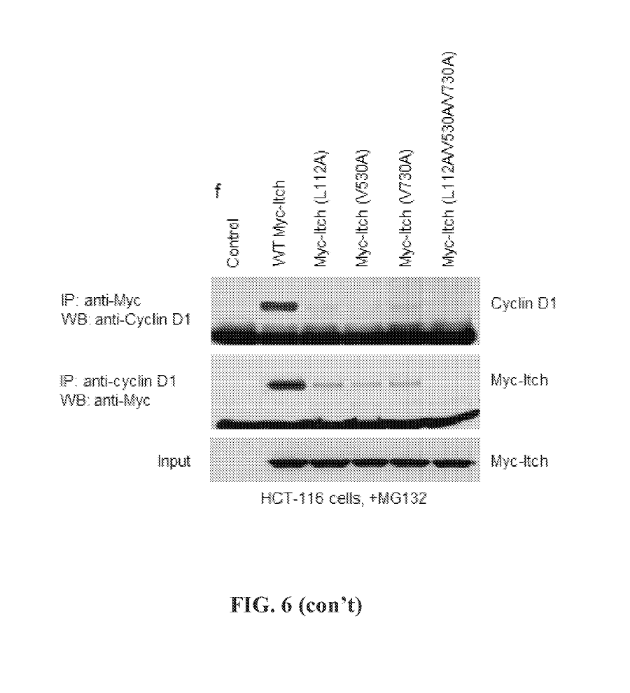

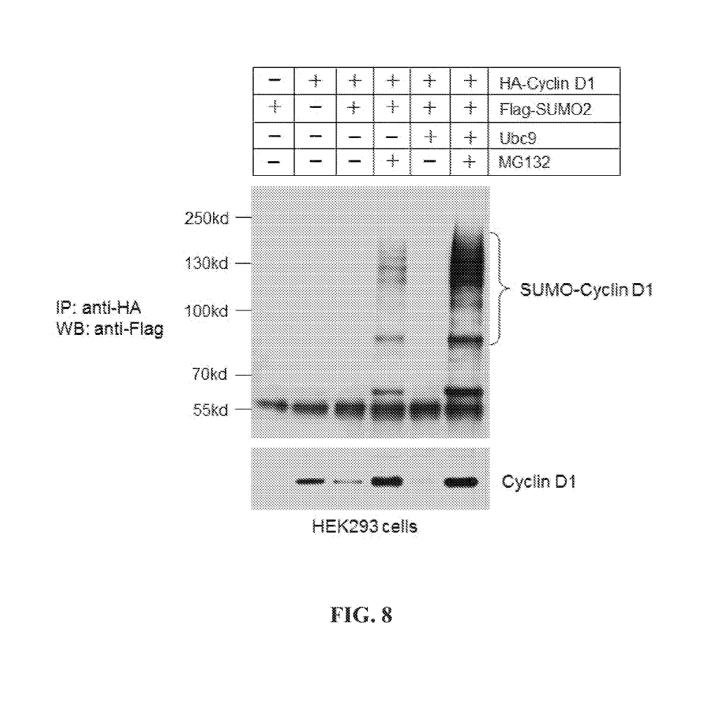

Previous studies indicate that phosphorylation of cyclin D1 leads to its degradation through ubiquitination mediated by multiple cullin-associated ubiquitin ligases during normal cell cycle progression. Cyclin D1 derivative bearing a threonine-to-alanine substitution at 286 (T286A) cannot be regulated by the cullin associated-E3 ligases (9, 10). However, our data showed that although this cyclin D1 mutant exhibits longer half-life compared with that of wild-type (WT) cyclin D1, it still degrades in the cells after treatment with cycloheximide (50 μg/ml) (FIG. 4a). Besides, poly-ubiquitination of mutant cyclin D1 (T286A) was detected (FIG. 6d) (11). These results indicate that in addition to phosphorylation, there should be other mechanism leading to ubiquitin-proteasome degradation involved in regulation of cyclin D1 protein level. SUMOylation is a post-translational modification process which is similar to ubiquitination. Genetic and proteomic evidences show that SUMO (Small Ubiquitin-related Modifier) target proteins participate in a variety of biological processes, essential to embryonic patterning, response to stress and cell cycle control (12-14). Recent studies unveiled the crosstalk between SUMO and ubiquitin pathways. A series of target proteins which are modified with multiple SUMOs can be recognized and polyubiquitinated, then subsequently result in proteasomal degradation (15). To investigate whether SUMOylation is involved in cyclin D1 degradation, we analyzed cyclin D1 expression by Western blot analysis after ectopic expression of Ubc9 or SUMO1/2/3 with or without proteasome inhibitor MG132. FIG. 1a shows that after expression of SUMO-conjugating enzyme Ubc9, the level of cyclin D1 markedly declined. Silencing of Ubc9 in human colon cancer cells HCT-116 resulted in an increase in cyclin D1 protein levels (FIG. 1e). Consistent with this finding we also observed that SUMO-specific protease 1, SENP1, blocked the inhibitory effect of Ubc9 on cyclin D1 degradation. Moreover, addition of proteasome inhibitor, MG132, also reversed the degradation of cyclin D1 caused by Ubc9 (FIG. 1a). Similar phenomena were also observed in experiments when SUMO1, 2 or 3 was overexpressed (FIG. 1b, 1c, 1d). These results indicate that SUMO pathway may be involved in mediating cyclin D1 degradation through the ubiquitin-proteasome system. Furthermore, results from co-immunoprecipitation assay showed that Ubc9 induced cyclin D1 protein SUMOylation as well as poly-ubiquitination in the presence of MG132; meanwhile, co-expression of SENP1 reversed this effect (FIG. 1f & FIG. 8). Collectively, these results suggest that multiple SUMO enzymes are involved in cyclin D1 SUMOylation which triggers cyclin D1 ubiquitination and proteosome degradation.

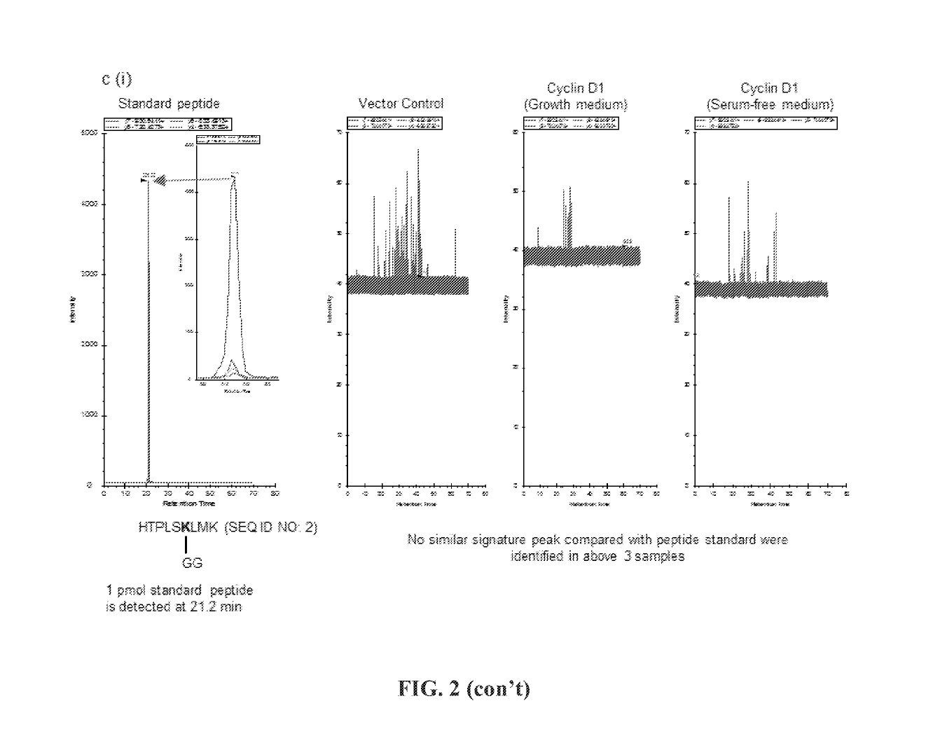

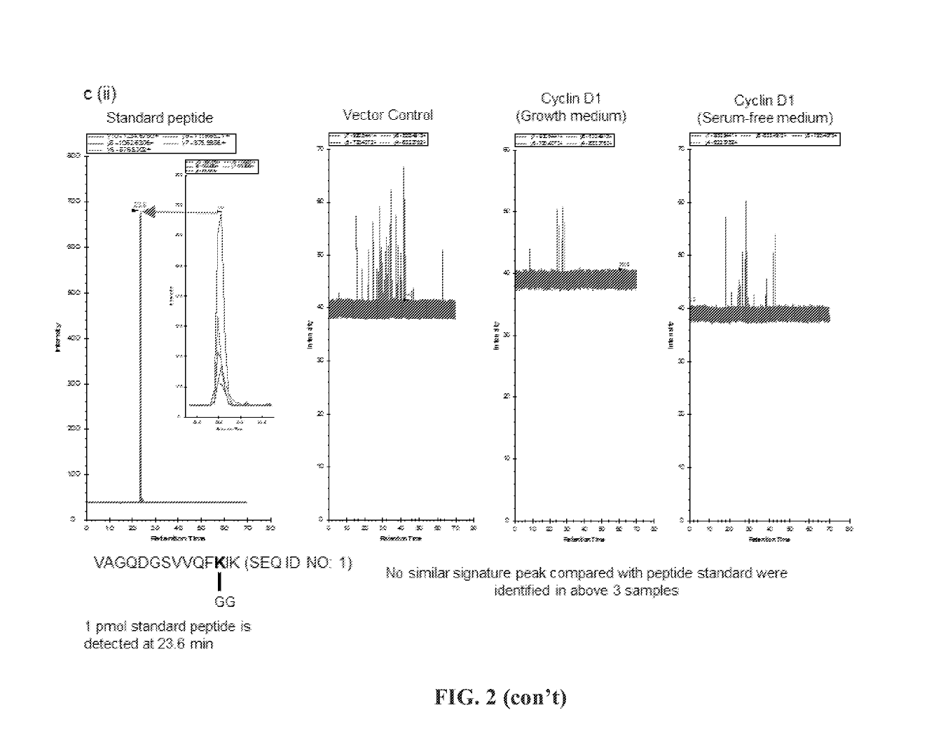

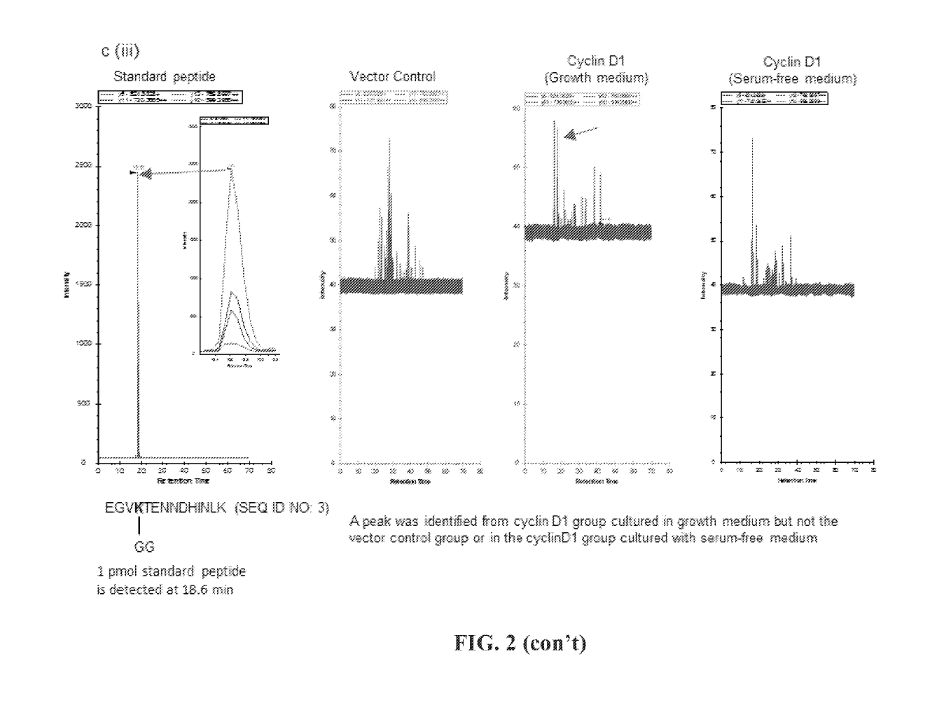

To further confirm this modification pattern of cyclin D1 protein, we performed the mass spectrometry. Through bioinformative prediction of potential ubiquitination sites on human SUMO-2 (NP_{—}008868.3) (16), we synthesized three peptides containing lysine 11, 32 or 41, respectively (FIG. 2a). The HA-tagged cyclin D1 conjugates were purified (FIG. 2b) and then analyzed by mass spectrometry under the LC-MRM mode (17) (FIG. 2a). Through comparing the specific peaks of the standard samples SEQ ID NOS.: 1-3, we found that ubiquitination of SUMO-2 on lysine 11 in the cells transfected with cyclin D1 but not in vector control cells (FIG. 2c). In addition, this modification can be only detected in the transfected cells cultured with normal growth medium with 10% fetal bovine serum. In contrast, no signal could be detected in the cells cultured with serum-free medium. We detected the cell cycle progression of the serum-free medium-cultured cells and found that the cells were synchronized before G1 phase (data not shown). Considering the fact that cyclin D1 degradation occurs mainly after G1 to S phase transition (18), our results suggest that this SUMOylation of cyclin D1 may exist during normal cell cycle progression.

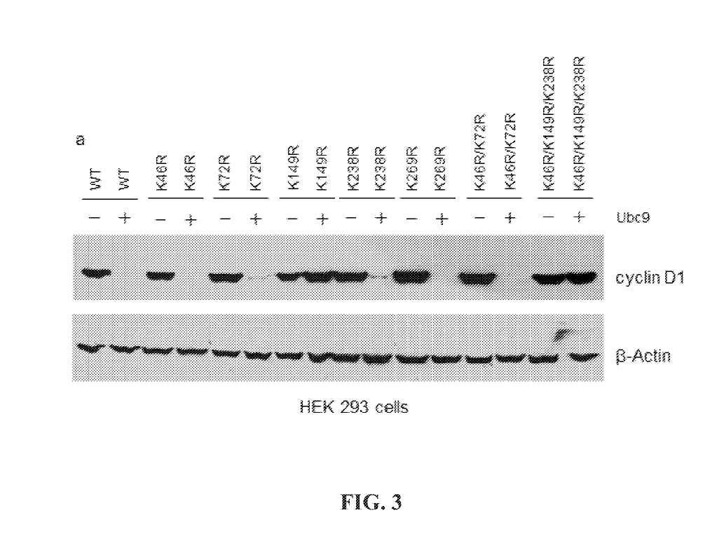

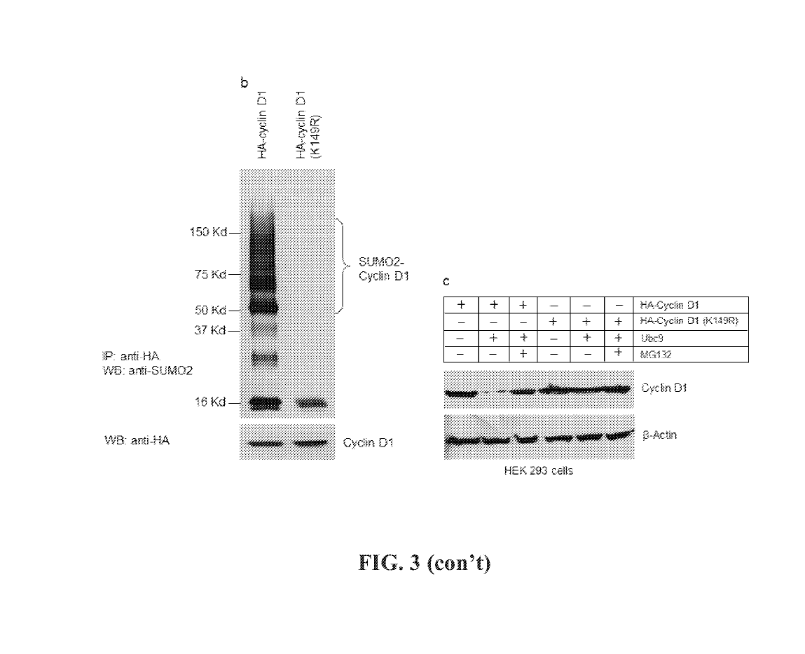

We next characterized the critical site for mediating cyclin D1 SUMOylation. Through analyzing cyclin D1 protein sequence (NP_{—}444284.1), a series of potential SUMOylation sites were found in this protein. Through site-directed mutagenesis, these sites were mutated individually, and lysine 149 turn to be the critical site for cyclin D1 SUMOylation. Cyclin D1 derivative bearing a lysine-to-arginine substitution at 149 (cyclin D1 (K149R)) was unaffected by ectopic Ubc9 expression (FIG. 3a&c). To further establish the SUMO-binding properties of cyclin D1, in vitro SUMOylation assay were performed. The result showed that compared with the wt cyclin D1, Cyclin D1 (K149R) lost the potential that can be modified with SUMOs (FIG. 3b).

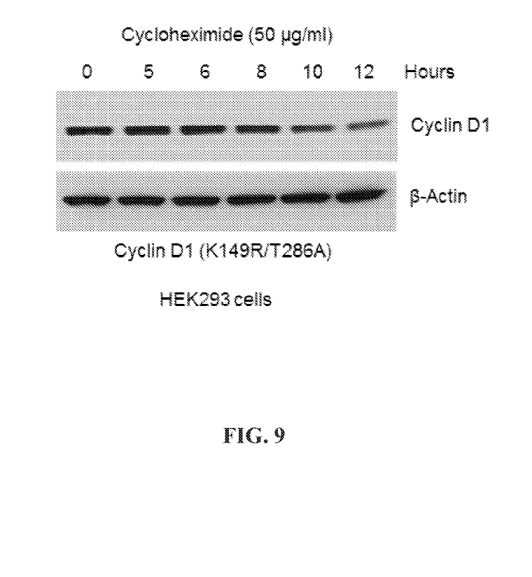

To further confirm the SUMO-modification of cyclin D1, we generated a mutant form of cyclin D1 in which both SUMOylation site (lysine 149) and phosphorylation site (threonine 286) were mutated (cyclin D1 (K149R/T286A). Results from protein decay assay showed that half-life of mutant cyclin D1 (K149R) or cyclin D1 (T286A) was longer than that of the WT cyclin D1 (FIG. 4a). Double mutant form of cyclin D1 is the most stable one among these four cyclin D1 constructs. Its expression kept stable for as long as 8 h in the presence of cycloheximide (50 μg/ml) (FIG. 4a; FIG. 9). Consistently we also found that cyclin D1 double mutant cannot be degraded by Ubc9 through SUMOylation-dependent ubiquitination or by DDB2 through phosphorylation-dependent ubiquitination (FIG. 4b). Results of luciferase assay also demonstrated that cells transfected with cyclin D1 double mutant had the highest activity on stimulating E2F-luc reporter comparing to the cells transfected with WT or cyclin D1 single mutant constructs (FIG. 4c). These results indicate that SUMOylation and phosphorylation are two critical mechanisms controlling cyclin D1 ubiquitination and proteasome degradation.

### Example 3

### SUMOylation Regulates Cyclin D1 Activity During Normal Cell Cycle Progression

Cyclin D1 functions as a critical cyclin during normal cell cycle progression, mainly during G1 to S phase transition (19). Functioning together with CDK4/6, cyclin D1 participates in mediating the phosphorylation of retinoblastoma protein, which results in the release of transcription factor E2F (20). E2F then transfers into nucleus and stimulates expression of a series of target genes, such as cyclin E and c-Myc, which are critical for the next step of cell cycle progression (21-23). It has been demonstrated that cyclin D1 protein level varies during the cell cycle progression. Highly expression of cyclin D1 is required for G1 to S phase transition. Once the cells have passed through the G1 phase and entered into the S phase, the cyclin D1 protein needs to be degraded (24). Phosphorylation-dependent cyclin D1 degradation occurs mainly during S phase (25).

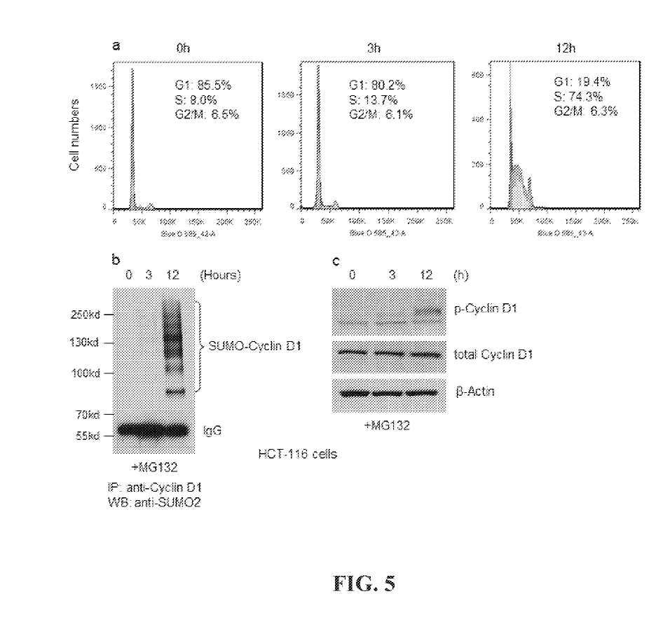

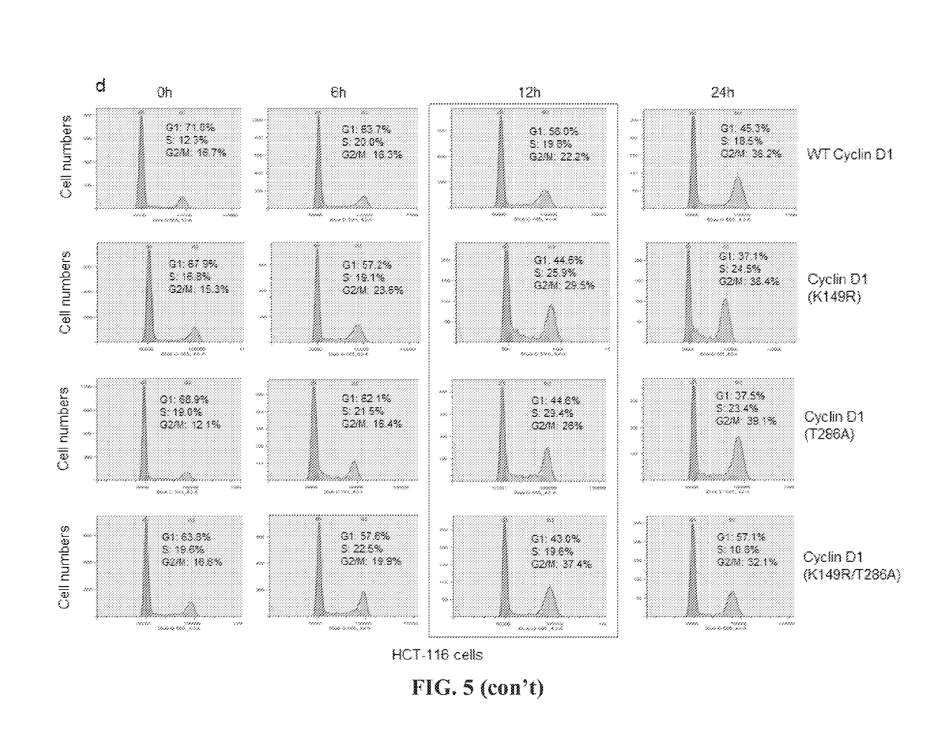

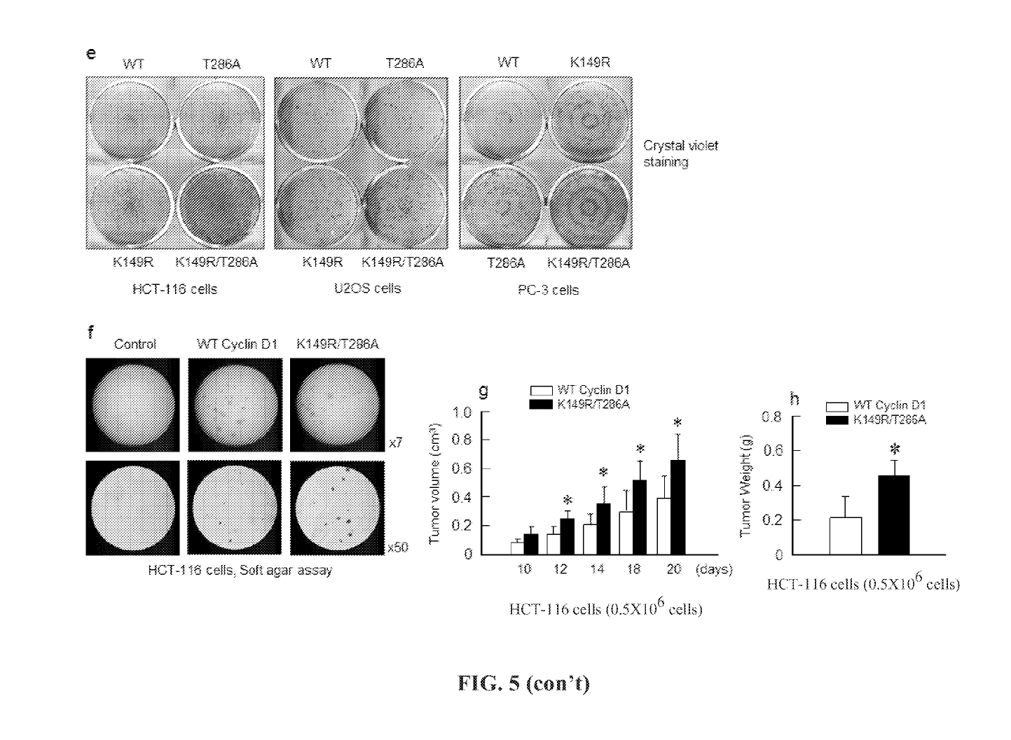

To test whether SUMOylation of cyclin D1 occurs during normal cell cycle progression, we performed in vivo SUMOylation assay. Human colon cancer cell line HCT-116 cells were blocked before G1 phase through overnight serum starvation. Endogenous SUMOylated-cyclin D1 as well as phosphorylated-cyclin D1 were detected through co-immunoprecipitation assays at different time points. Results of flow cytometry showed that 12 h after the cells were released into cell cycle, most of the cells (74.3%) had already passed through G1 phase and entered into S-phase (FIG. 5a), and this was accompanied by an increase in cyclin D1 SUMOylation (FIG. 5b). As a control, we also found that cyclin D1 phosphorylation was also increased dramatically at 12 h time point, which is consistent with the previous reports (25) (FIG. 5c). These results indicate that similar to phosphorylation-dependent degradation, SUMOylation of cyclin D1 is another modification mechanism that regulates cyclin D1 protein levels during normal cell cycle G1-S transition.

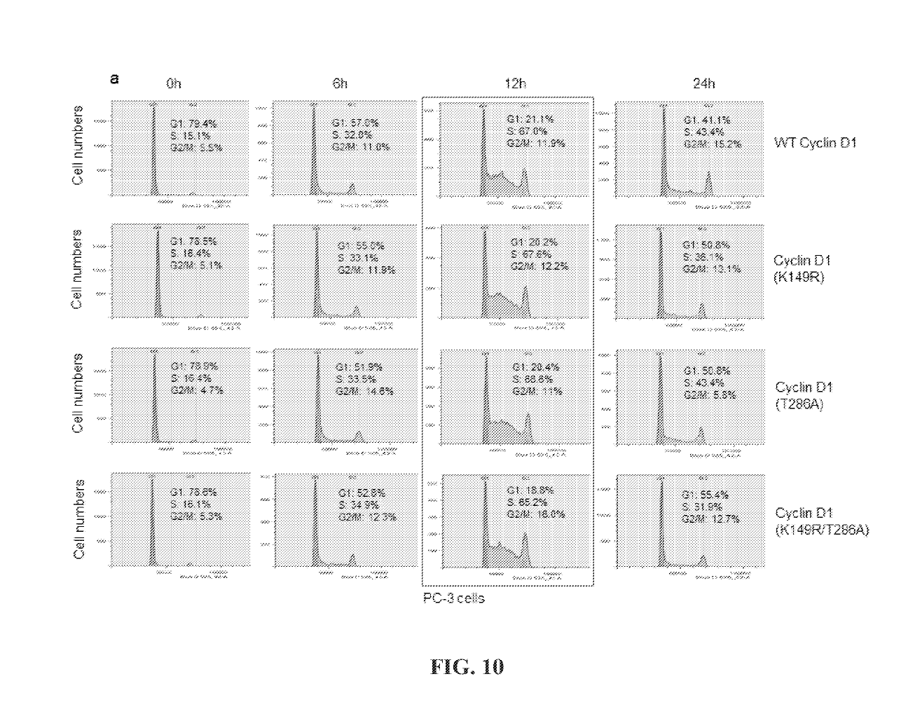

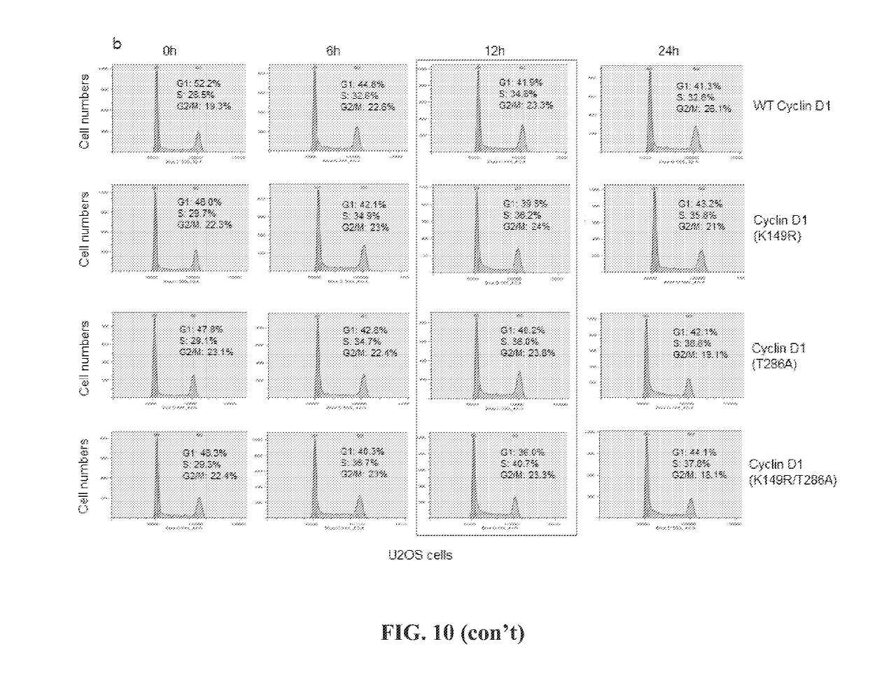

To further confirm this result, WT and three mutant forms of cyclin D1 (K149R, T286A and K149R/T286A) constructs were stably transfected into three types of human cancer cells, HCT-116, U2OS (human osteosarcoma cells), PC-3 (human prostate cancer cells), respectively. The cells were synchronized before G1 phase through serum-starvation, and then subsequently released into normal cell cycle. As FIG. 5d shown, 12 h after the release, cell progression rate of PC-3 cells stably transfected with cyclin D1 double mutant (K149R/T286A) turned to be much faster than those cells transfected with WT or single mutant forms of cyclin D1. Similar results were obtained in the other two types of cancer cells (FIG. 10a&10b). Moreover, these three human cancer cells (HCT-116, U205, PC-3) stably transfected with cyclin D1 double mutant exhibited much more accelerated growth rate than the other groups (FIG. 5e). Based on the observation that cyclin D1 double mutant is resistant to ubiquitin-dependent proteolysis and facilitates cell growth, we performed a colony formation assay in soft agar to test whether cyclin D1 double mutant regulates the anchorage-independent growth of HCT-116 cells. Stable transfection of cyclin D1 double mutant construct increased both colony number and size compared with WT cyclin D1 transfected group (FIG. 5f).

Since inhibition of SUMOylation and phosphorylation of cyclin D1 accelerates cell growth and increase cell transformation in vitro, we then determined if double mutant cyclin D1 promotes tumor cell growth in vivo using a flank allograft model. HCT-116 cells stably transfected with WT or cyclin D1 double mutant were grafted into athymic nude mice and then tumor growth measured by tumor weight was examined. FIG. 5g&5 h showed that ectopic expression of cyclin D1 double mutant resulted in more accelerated growth rate than the cells transfected with WT cyclin D1.

### Example 4

### Itch Specifically Ubiquitinates SUMOylated Cyclin D1

Recent proteomic studies using cells isolated from Flag-cyclin D1 knockin mice and high-throughput mass spectrometry approach identified interaction of Itch with cyclin D1, suggesting that Itch is a critical endogenous E3 ligase regulating cyclin D1 degradation (26). Itch, also named as atrophin-1 interacting protein 4 (AIP4), belongs to HECT-domain E3 ligase and is different from the F-box E3 ligases which have been reported to be involved in phosphorylation-dependent cyclin D1 degradation. Itch knockout mice have a severe autoimmune phenotype (27). In this study, we examined the role of Itch in SUMOylation-mediated cyclin D1 degradation. We found that steady-state protein levels of cyclin D1 were increased in most tissues in Itch knockout mice (FIG. 6a). Ectopic expression of Itch dramatically reduced cyclin D1 levels, while this effect was blocked in the presence of SUMO-2 siRNA or Ubc9 siRNA, suggesting that Itch mediates cyclin D1 degradation in a SUMOylation-dependent manner (FIG. 6b). We also found that addition of MG132 also reversed the effect of Itch on cyclin D1 degradation, suggesting that proteasome degradation is also involved in this process (FIG. 6c). Interestingly, Itch remains active on the ubiquitination and proteasome degradation of phosphorylation mutant form of cyclin D1 (T286A) (FIG. 6c&d). These results rule out the possibility that Itch is involved in phosphorylation-dependent cyclin D1 degradation. In contrast, Itch had no effect on the ubiquitination of SUMOylation mutant form of cyclin D1 (K149R) (FIG. 6d). Seven putative SUMO Interacting Motifs (SIMs) were identified in Itch protein through sequence analysis. To determine the interacting motif(s) of Itch recognizing SUMOylated cyclin D1 protein, we generated a series of mutants of Itch. We found that Itch completely lost its ability to induce cyclin D1 degradation when three potential SIMs were mutated (L112A/V530A/V731A), (FIG. 6e). These results were further confirmed by co-immunoprecipitation assay showing that mutant Itch (L112/V530/V731A) could not interact with cyclin D1 any more (FIG. 6f). These findings indicate that Itch functions as a specific E3 ligase to mediate SUMOylated cyclin D1 ubiquitination and proteasome degradation.

### Example 5

### Arsenic Trioxide Mediates Cyclin D1 Degradation in a SUMOylation-Dependent Manner

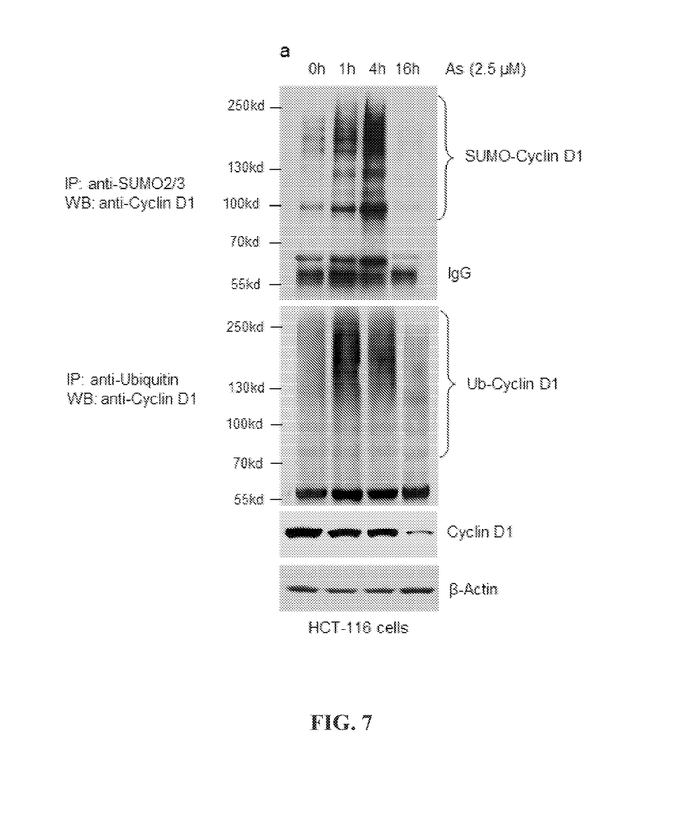

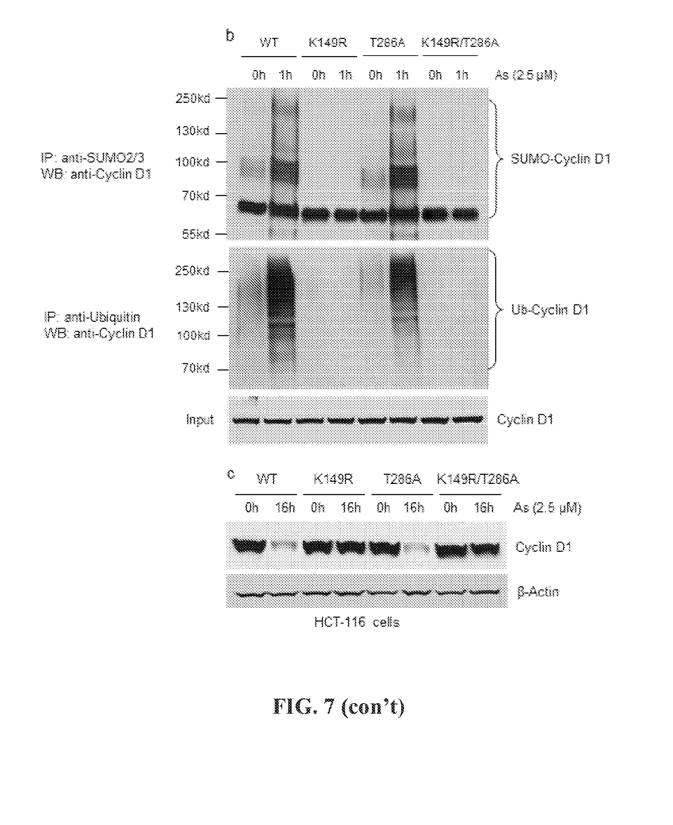

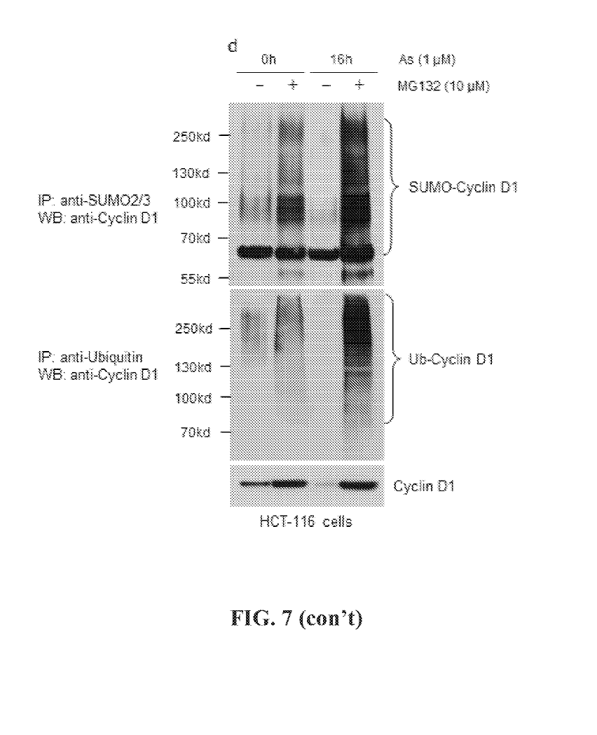

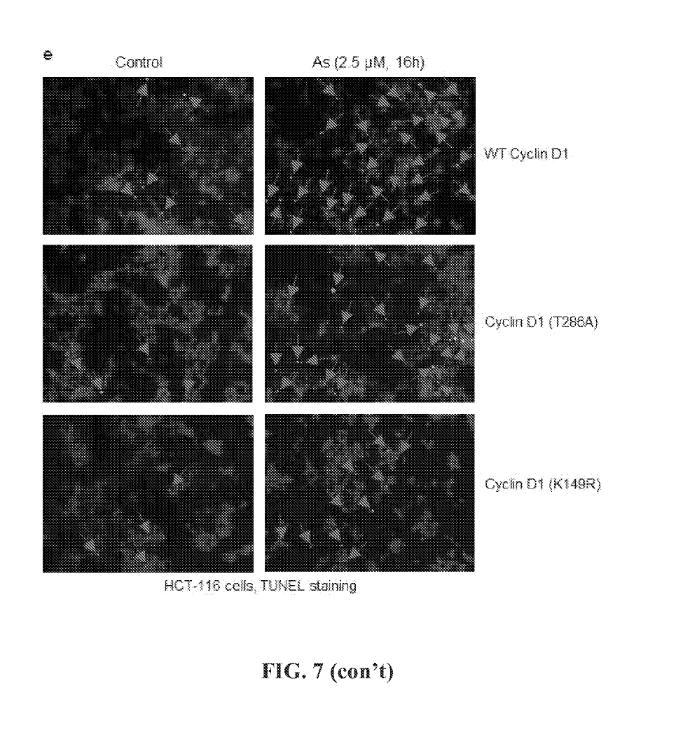

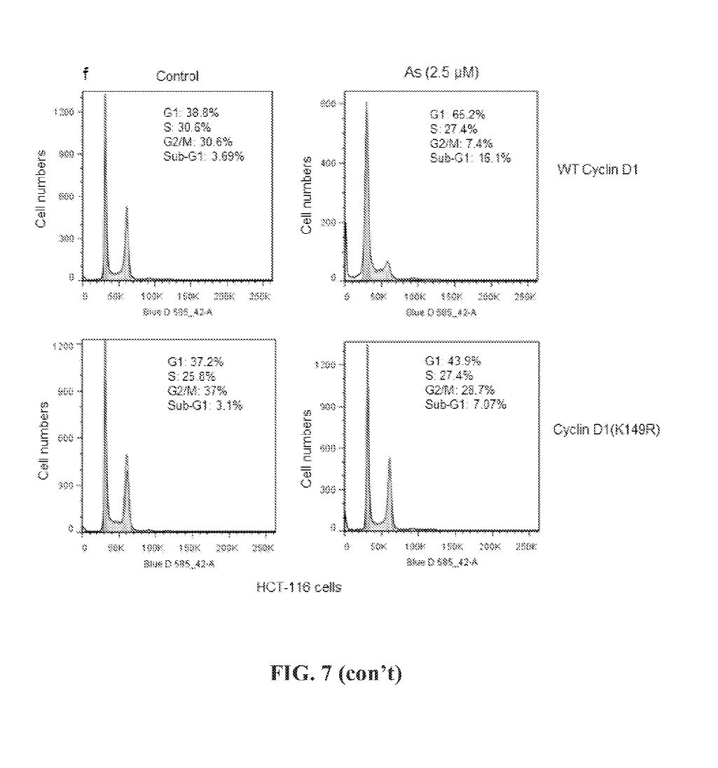

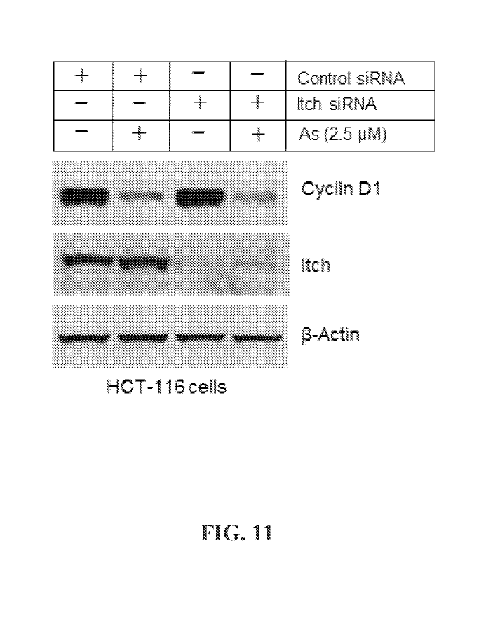

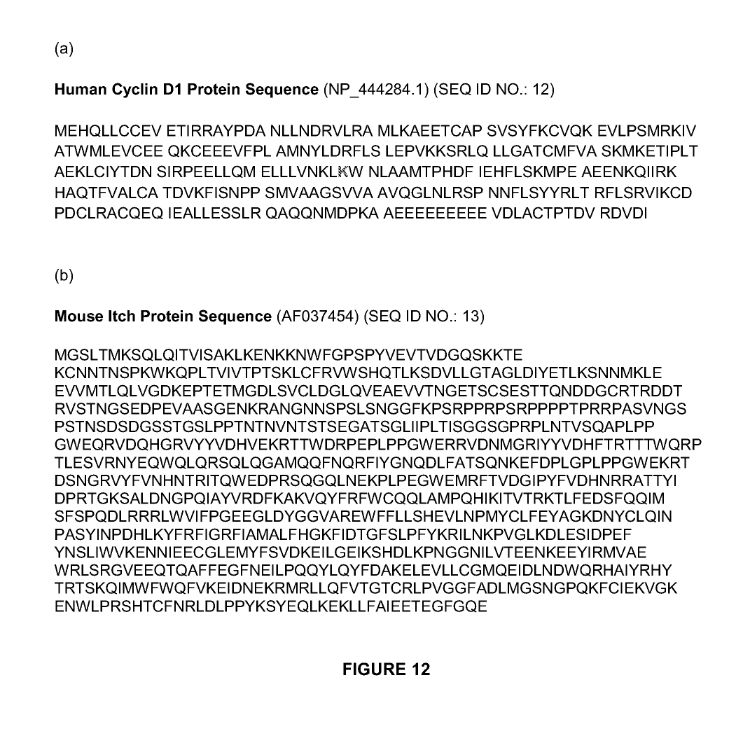

As a proto-oncogene, cyclin D1 gene amplification as well as protein overexpression has been found in many kinds of human cancers (4-8). To determine if cyclin D1 could serve as a target for cancer treatment, we examined the role of arsenic trioxide (As₂O₃) in cyclin D1 SUMOylation and degradation. As₂O₃, despite of its well-known toxicity, has been used for cancer treatment in traditional Chinese medicine for a long time (28, 29). As₂O₃ functions to disrupt the metabolic system of cells through allosteric inhibition of pyruvate dehydrogenase complex (30, 31). Several studies demonstrated that this compound induces cancer cell apoptosis as well as cell cycle arrest at the G1 phase (32, 33). Recently, several groups found that As₂O₃ could target a fusion oncoprotein, PML-RARα. As₂O₃ directly binds with PML which induces the conformational change of PML leading to the SUMOylation of PML protein (16, 29, 34). In the present studies, we found cyclin D1 is a new target protein for As₂O₃ and As₂O₃ could induce cyclin D1 degradation in a SUMOylation-dependent manner. We treated HCT-116 cells with As₂O₃ for 16 h and found that SUMOylated- as well as polyubiquitinated-cyclin D1 was accumulated 1 h after As₂O₃ treatment (FIG. 7a). After 16 h treatment, modified cyclin D1 disappeared due to its degradation (FIG. 7a). These results indicate that As₂O₃ may induce cyclin D1 degradation through SUMOylation pathway. To further determine whether phosphorylation of cyclin D1 is also involved during this process, WT and mutant cyclin D1 (K149R, T286A, and K149R/T286A) were stably expressed in HCT-116 cells and the cells were treated with As₂O₃ for 1 and 16 hours. As₂O₃ induced SUMOylation, polyubiquitination and degradation of cyclin D1 in the cells transfected with WT or T286A mutant cyclin D1, indicating As₂O₃-mediated cyclin D1 degradation is phosphorylation-independent. In contrast, As₂O₃ had no effect on the degradation of K149R or K149R/T286A mutant forms of cyclin D1 (FIG. 7b&c), indicating that As₂O₃-mediated cyclin D1 degradation is SUMOylation-dependent. Besides, As₂O₃-mediated cyclin D1 SUMOylation and polyubiquitination can be reversed by addition of proteasome inhibitor MG132 (FIG. 7d). This result further confirmed that proteasome system is involved in As₂O₃ induced-cyclin D1 degradation. In HCT-116 cells, silencing of Itch expression did not abrogated degradation of cyclin D1 induced by As₂O₃ treatment (FIG. 11), indicating that other ubiquitin ligases are involved in this process. To further study the mechanism of As₂O₃-induced cancer cells apoptosis, HCT-116 cells were stably transfected with WT and mutant (K149R and T286A) cyclin D1 constructs and treated with As₂O₃ (2.5 μM). Results of TUNNEL staining showed that treatment with As₂O₃ induced accelerated cell apoptosis in the cells stably transfected with WT and T286A cyclin D1. In contrast, the effect of As₂O₃ on cell apoptosis was much lower in the cells stably transfected with K149R mutant cyclin D1 (FIG. 7e). Results from flow cytometry also showed that As₂O₃ failed to induce efficient G1 arrest in the cells transfected with K149R mutant cyclin D1 compared with cells transfected with WT cyclin D1 (FIG. 7f). These results indicate that arsenic trioxide mediates cancer cell apoptosis and induces G1 arrest partially through inducing cyclin D1 degradation in a SUMOylation-dependent manner.

### Example 6

### Summary of Examples 2-5

Cyclin D1 is SUMOylated and is subsequently ubiquitinated and proteasome degraded. We have identified the SUMOylation site of cyclin D1 and found that lysine 149 of cyclin D1 is the sumoylation site. Cyclin cannot be SUMOylated when lysine 149 of cyclin D1 is mutated (K149R). We have identified a specific E3 ligase (Itch), which recognizes the SUMOylated cyclin D1. We have mapped SUMO-interacting motif (SIM) of Itch protein. We have demonstrated that cyclin D1 SUMOylation mainly occurs at the S phase of the cell cycle. Mutation of cyclin D1 (K149R) inhibits cyclin D1 SUMOylation and promotes cell cycle G1/S transition. Inoculation of tumor cells (HCT-116 colon cancer cells) expressing mutant cyclin D1 (K149R) into nude mice promotes tumor growth compared to the nude mice inoculated with tumor cells expressing wild-type cyclin D1. Arsenic trioxide induces cyclin D1 SUMOylation and ubiquitination.

We have identified a novel mechanism of cancer development (i.e., defects in cyclin D1 SUMOylation). We have identified novel drug targets such as Ubc9 (i.e., a SUMO E2 enzyme) and Itch (i.e., E3 ligase, recognizing SUMOylated cyclin D1). We have identified a novel agent to treat cancer (i.e., arsenic trioxide, which induces cyclin D1 SUMOylation).

### Example 7

### Discussion of Examples 2-5

In summary, our current study demonstrates a novel mechanism controlling cyclin D1 post-translational regulation. Cyclin D1 can be recognized by multiple SUMO proteins leading to its ubiquitin-proteasome degradation. Similar to phosphorylation, SUMOylation of cyclin D1 also occurs during normal cell cycle progression, mainly during G1-S transition phase. We have determined the critical SUMOylation site, lysine 149, on cyclin D1 protein. Once this site is mutated into arginine, cyclin D1 cannot be modified through SUMOylation. We found that Itch functions as a specific E3 ligase interacting with SUMOylated-cyclin D1 and mediates cyclin D1 ubiquitination. Itch induces cyclin D1 degradation through the proteasome system. Mutations of three SIMs on Itch protein (L112A/V530A/V731A) completely abolished the interaction of Itch with cyclin D1. We also found that As₂O₃ triggers cyclin D1 proteasomal degradation in a SUMOylation-dependent manner. This regulatory mechanism may significantly contribute to As₂O₃-induced cancer cell apoptosis.

In eukaryocytes, SUMOylation functions as a three-step post-translational modification process similar to ubiquitination. SUMO pathway controls many aspects of protein functions, such as subcellular localization (35), transactivation of transcription factors (36,37) and DNA repair (38). Recent studies found that this modification process also participates in regulation of cell cycle progression. It has been reported that septins are modified with SUMOs specifically during mitosis in S. cerevisiae (39). SUMO-specific protease SENP5 is required for cell division⁴⁰. In fact, before the SUMO pathway has been clearly characterized, Ubc9 was found to regulate the activity of cyclins and play a critical role in S- and M-phase cell cycle progression. In Ubc9 loss-of-function mutant, a series of cell cycle proteins, including CLB2/5, cyclin A, and cyclin B, are stabilized (41), although the mechanism is unknown. Our studies provide novel evidence for Ubc9 function as the E2 conjugating enzyme during SUMOylation and induces the proteolysis of cyclins, such as cyclin D1 (or possibly other cyclins), through SUMOylation-dependent mechanism. The modification of cyclin D1 with SUMOs occurs during normal cell cycle progression and this mechanism regulates the cyclin D1 stability and controls the rate of cell division. Thus, we have demonstrated for the first time that cyclin D1 is the target of SUMO pathway during cell cycle regulation.

In our study, we found that phosphorylation and SUMOylation mutant cyclin D1 is the most stable and active form of cyclin D1. The relationship between these two regulatory mechanisms needs to be further investigated. Our data demonstrate that there is no significant difference about the phosphorylation status between WT and K149R mutant cyclin D1. In addition, both WT and T286A mutant cyclin D1 can be SUMOylated (FIG. 7a). Taken together, our study suggests that SUMOylation is another important regulatory mechanism controlling cyclin D1 protein stability during normal cell cycle progression.

Our study also suggests defects in cyclin D1 SUMOylation may lead to cell transformation and tumorigenesis. In fact, it has been reported that loss of control on SUMOylation or deSUMOylation process could result in defects in the maintenance of cell homeostasis and lead to cancer development (42). In normal cells, SUMO pathway participates in the induction of cell senescence in a p53- and Rb-dependent manner (43). However, this process is blocked in cancer cells which possess mutations of these two tumor suppressor genes (44). SENP1 up-regulation has been found in thyroid and prostate cancers and this overexpression facilitates neoplastic development in the prostate (45,46). SENP3 is found with increased stability through interacting with Hsp90 in hepatoma patient samples (47). These findings suggest that protein SUMOylation could be used as a potential target for future cancer treatment. Arsenic trioxide has been found to induce SUMOylation-dependent proteolysis of oncoprotein PML (29). This compound induces cell apoptosis in both solid and liquid tumors (48,49,50,51) and results in tumor shrink in nude mice (52). There are several explanations for how arsenic trioxide functions to induce cell apoptosis, such as inducing polymerization of microtubules (51), antagonizing the Hedgehog pathway (52) or modifying cell cycle progress (53). However, the detailed mechanism remains unknown. Our studies demonstrate that cyclin D1 is a target protein of arsenic trioxide. This compound induces cell apoptosis partially through inducing cyclin D1 degradation in a SUMOylation-dependent manner. Once the SUMOylation site of cyclin D1 is mutated, the effect of arsenic trioxide on tumor cell apoptosis was significantly decreased. Our studies provide novel mechanism by which arsenic trioxide regulates cancer cell apoptosis.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

### 5. REFERENCES

1 Tam, S. W., Theodoras, A. M., Shay, J. W., Draetta, G. F. & Pagano, M. Differential expression and regulation of Cyclin D1 protein in normal and tumor human cells: association with Cdk4 is required for Cyclin D1 function in G1 progression. Oncogene 9, 2663-2674 (1994).
2 Alao, J. P. The regulation of cyclin D1 degradation: roles in cancer development and the potential for therapeutic invention. Mol Cancer 6, 24, doi:10.1186/1476-4598-6-24 (2007).
3 Klein, E. A. & Assoian, R. K. Transcriptional regulation of the cyclin D1 gene at a glance. J Cell Sci 121, 3853-3857, doi:10.1242/jcs.039131 (2008).
4 Bartkova, J. et al. Cyclin D1 protein expression and function in human breast cancer. Int J Cancer 57, 353-361 (1994).
5 Betticher, D. C. et al. Prognostic significance of CCND1 (cyclin D1) overexpression in primary resected non-small-cell lung cancer. Br J Cancer 73, 294-300 (1996).
6 Gautschi, O., Ratschiller, D., Gugger, M., Betticher, D. C. & Heighway, J. Cyclin D1 in non-small cell lung cancer: a key driver of malignant transformation. Lung Cancer 55, 1-14, doi:10.1016/j.lungcan.2006.09.024 (2007).
7 Drobnjak, M., Osman, I., Scher, H. I., Fazzari, M. & Cordon-Cardo, C. Overexpression of cyclin D1 is associated with metastatic prostate cancer to bone. Clin Cancer Res 6, 1891-1895 (2000).
8 Wagner, U. et al. Cyclin D1 overexpression lacks prognostic significance in superficial urinary bladder cancer. J Pathol 188, 44-50, doi:10.1002/(SICI)1096-9896(199905)188:1<44::AID-PATH320>3.0.CO;2-Q (1999).
9 Lin, D. I. et al. Phosphorylation-dependent ubiquitination of cyclin D1 by the SCF (FBX4-alphaB crystallin) complex. Mol Cell 24, 355-366, doi:10.1016/j.molcel.2006.09.007 (2006).
10 Okabe, H. et al. A critical role for FBXW8 and MAPK in cyclin D1 degradation and cancer cell proliferation. PLoS One 1, e128, doi:10.1371/journal.pone.0000128 (2006).
11 Germain, D., Russell, A., Thompson, A. & Hendley, J. Ubiquitination of free cyclin D1 is independent of phosphorylation on threonine 286. J Biol Chem 275, 12074-12079 (2000).
12 Wang, Y. & Dasso, M. SUMOylation and deSUMOylation at a glance. J Cell Sci 122, 4249-4252, doi:10.1242/jcs.050542 (2009).
13 Wilkinson, K. A. & Henley, J. M. Mechanisms, regulation and consequences of protein SUMOylation. Biochem J 428, 133-145, doi:10.1042/BJ20100158 (2010).
14 Sarge, K. D. & Park-Sarge, O. K. Sumoylation and human disease pathogenesis. Trends Biochem Sci 34, 200-205, doi:10.1016/j.tibs.2009.01.004 (2009).
15 Hunter, T. & Sun, H. Crosstalk between the SUMO and ubiquitin pathways. Ernst Schering Found Symp Proc, 1-16 (2008).
16 Tatham, M. H. et al. RNF4 is a poly-SUMO-specific E3 ubiquitin ligase required for arsenic-induced PML degradation. Nat Cell Biol 10, 538-546, doi:10.1038/ncb1716 (2008).
17 Wang, Q. et al. Mutant proteins as cancer-specific biomarkers. Proc Natl Acad Sci USA 108, 2444-2449, doi:10.1073/pnas.1019203108 (2011).
18 Diehl, J. A., Cheng, M., Roussel, M. F. & Sherr, C. J. Glycogen synthase kinase-3beta regulates cyclin D1 proteolysis and subcellular localization. Genes Dev 12, 3499-3511 (1998).
19 Baldin, V., Lukas, J., Marcote, M. J., Pagano, M. & Draetta, G. Cyclin D1 is a nuclear protein required for cell cycle progression in G1. Genes Dev 7, 812-821 (1993).
20 Zhu, X., Ohtsubo, M., Bohmer, R. M., Roberts, J. M. & Assoian, R. K. Adhesion-dependent cell cycle progression linked to the expression of cyclin D1, activation of cyclin E-cdk2, and phosphorylation of the retinoblastoma protein. J Cell Biol 133, 391-403 (1996).
21 Ohtani, K., DeGregori, J. & Nevins, J. R. Regulation of the cyclin E gene by transcription factor E2F1. Proc Natl Acad Sci USA 92, 12146-12150 (1995).
22 Kel, A. E. et al. Computer-assisted identification of cell cycle-related genes: new targets for E2F transcription factors. J Mol Biol 309, 99-120, doi:10.1006/jmbi.2001.4650 (2001).
23 Nevins, J. R. The Rb/E2F pathway and cancer. Hum Mol Genet 10, 699-703 (2001).
24 Yang, K., Hitomi, M. & Stacey, D. W. Variations in cyclin D1 levels through the cell cycle determine the proliferative fate of a cell. Cell Div 1, 32, doi:10.1186/1747-1028-1-32 (2006).
25 Guo, Y. et al. Phosphorylation of cyclin D1 at Thr 286 during S phase leads to its proteasomal degradation and allows efficient DNA synthesis. Oncogene 24, 2599-2612, doi:10.1038/sj.onc.1208326 (2005).
26 Bienvenu, F. et al. Transcriptional role of cyclin D1 in development revealed by a genetic-proteomic screen. Nature 463, 374-378, doi:10.1038/nature08684 (2010).
27 Parravicini, V., Field, A. C., Tomlinson, P. D., Basson, M. A. & Zamoyska, R. Itch−/− alphabeta and gammadelta T cells independently contribute to autoimmunity in Itchy mice. Blood 111, 4273-7282, doi:10.1182/blood-2007-10-115667 (2008).
28 Siu, K. P., Chan, J. Y. & Fung, K. P. Effect of arsenic trioxide on human hepatocellular carcinoma HepG2 cells: inhibition of proliferation and induction of apoptosis. Life Sci 71, 275-285 (2002).
29 Zhang, X. W. et al. Arsenic trioxide controls the fate of the PML-RARalpha oncoprotein by directly binding PML. Science 328, 240-243, doi:10.1126/science.1183424 (2010).
30 Aposhian, H. V. & Aposhian, M. M. Arsenic toxicology: five questions. Chem Res Toxicol 19, 1-15, doi:10.1021/tx050106d (2006).
31 Samikkannu, T. et al. Reactive oxygen species are involved in arsenic trioxide inhibition of pyruvate dehydrogenase activity. Chem Res Toxicol 16, 409-414, doi:10.1021/tx025615j (2003).
32 Miller, W. H., Jr., Schipper, H. M., Lee, J. S., Singer, J. & Waxman, S. Mechanisms of action of arsenic trioxide. Cancer Res 62, 3893-3903 (2002).
33 Park, W. H. et al. Arsenic trioxide-mediated growth inhibition in MC/CAR myeloma cells via cell cycle arrest in association with induction of cyclin-dependent kinase inhibitor, p21, and apoptosis. Cancer Res 60, 3065-3071 (2000).
34 Lallemand-Breitenbach, V. et al. Arsenic degrades PML or PML-RARalpha through a SUMO-triggered RNF4/ubiquitin-mediated pathway. Nat Cell Biol 10, 547-555, doi:10.1038/ncb1717 (2008).
35 Sternsdorf, T., Jensen, K., Reich, B. & Will, H. The nuclear dot protein sp100, characterization of domains necessary for dimerization, subcellular localization, and modification by small ubiquitin-like modifiers. J Biol Chem 274, 12555-12566 (1999).
36 Bae, S. H. et al. Sumoylation increases HIF-1alpha stability and its transcriptional activity. Biochem Biophys Res Commun 324, 394-400, doi:10.1016/j.bbrc.2004.09.068 (2004).
37 Le Drean, Y., Mincheneau, N., Le Goff, P. & Michel, D. Potentiation of glucocorticoid receptor transcriptional activity by sumoylation. Endocrinology 143, 3482-3489 (2002).
38 Zhao, X. & Blobel, G. A SUMO ligase is part of a nuclear multiprotein complex that affects DNA repair and chromosomal organization. Proc Natl Acad Sci USA 102, 4777-4782, doi:10.1073/pnas.0500537102 (2005).
39 Johnson, E. S. & Blobel, G. Cell cycle-regulated attachment of the ubiquitin-related protein SUMO to the yeast septins. J Cell Biol 147, 981-994 (1999).
40 Di Bacco, A. et al. The SUMO-specific protease SENP5 is required for cell division. Mol Cell Biol 26, 4489-4498, doi:10.1128/MCB.02301-05 (2006).
41 Seufert, W., Futcher, B. & Jentsch, S. Role of a ubiquitin-conjugating enzyme in degradation of S- and M-phase cyclins. Nature 373, 78-81, doi:10.1038/373078a0 (1995).
42 Kim, K. I. & Baek, S. H. SUMOylation code in cancer development and metastasis. Mol Cells 22, 247-253 (2006).
43 Li, T. et al. Expression of SUMO-2/3 induced senescence through p53- and pRB-mediated pathways. J Biol Chem 281, 36221-36227, doi:10.1074/jbc.M608236200 (2006).
44 Bischof, O. et al. The E3 SUMO ligase PIASy is a regulator of cellular senescence and apoptosis. Mol Cell 22, 783-794, doi:10.1016/j.molcel.2006.05.016 (2006).
45 Cheng, J., Bawa, T., Lee, P., Gong, L. & Yeh, E. T. Role of desumoylation in the development of prostate cancer. Neoplasia 8, 667-676, doi:10.1593/neo.06445 (2006).
46 Jacques, C. et al. Two-step differential expression analysis reveals a new set of genes involved in thyroid oncocytic tumors. J Clin Endocrinol Metab 90, 2314-2320, doi:10.1210/jc.2004-1337 (2005).
47 Yan, S. et al. Redox regulation of the stability of the SUMO protease SENP3 via interactions with CHIP and Hsp90. Embo J 29, 3773-3786, doi:10.1038/emboj.2010.245 (2010).
48 Huang, X. J., Wiernik, P. H., Klein, R. S. & Gallagher, R. E. Arsenic trioxide induces apoptosis of myeloid leukemia cells by activation of caspases. Med Oncol 16, 58-64 (1999).
49 Shim, M. J. et al. Arsenic trioxide induces apoptosis in chronic myelogenous leukemia K562 cells: possible involvement of p38 MAP kinase. J Biochem Mol Biol 35, 377-383 (2002).
50 Shen, Z. Y. et al. Arsenic trioxide induces apoptosis of oesophageal carcinoma in vitro. Int J Mol Med 4, 33-37 (1999).
51 Ling, Y. H., Jiang, J. D., Holland, J. F. & Perez-Soler, R. Arsenic trioxide produces polymerization of microtubules and mitotic arrest before apoptosis in human tumor cell lines. Mol Pharmacol 62, 529-538 (2002).
52 Kim, J., Lee, J. J., Gardner, D. & Beachy, P. A. Arsenic antagonizes the Hedgehog pathway by preventing ciliary accumulation and reducing stability of the Gli2 transcriptional effector. Proc Natl Acad Sci USA 107, 13432-13437, doi:10.1073/pnas.1006822107 (2010).
53 Li, X., Ding, X. & Adrian, T. E. Arsenic trioxide induces apoptosis in pancreatic cancer cells via changes in cell cycle, caspase activation, and GADD expression. Pancreas 27, 174-179 (2003).
54 Zhang, M. et al. PTHrP prevents chondrocyte premature hypertrophy by inducing cyclin-D1-dependent Runx2 and Runx3 phosphorylation, ubiquitylation and proteasomal degradation. J Cell Sci 122, 1382-1389, doi:10.1242/jcs.040709 (2009).
55 Santra, M. K., Wajapeyee, N. & Green, M. R. F-box protein FBXO31 mediates cyclin D1 degradation to induce G1 arrest after DNA damage. Nature 459, 722-725, doi:10.1038/nature08011 (2009).
56 MacLean, B. et al. Skyline: an open source document editor for creating and analyzing targeted proteomics experiments. Bioinformatics 26, 966-968, doi:10.1093/bioinformatics/btq054 (2010).
57 Newman, R. M. et al. Antizyme targets cyclin D1 for degradation. A novel mechanism for cell growth repression. J Biol Chem 279, 41504-41511, doi:10.1074/jbc.M407349200 (2004).
58 Magnifico, A. et al. WW domain HECT E3s target Cbl RING finger E3s for proteasomal degradation. J Biol Chem 278, 43169-43177, doi:10.1074/jbc.M308009200 (2003).

1. A method for treating cancer, comprising administering to a subject in need of such treatment a composition comprising a therapeutically effective amount of an agent that mediates downregulation of cyclin D1.

2. The method of claim 1, wherein the cancer is selected from the group consisting of breast cancer, lung cancer, prostate cancer, and bladder cancer.

3. The method of claim 1, wherein the agent mediates downregulation of cyclin D1 by increasing sumoylation of cyclin D1.

4. The method of claim 3, wherein the agent is arsenic trioxide.

5. The method of claim 3, wherein the agent upregulates activity of at least one of an E3 ligase and a SUMO-conjugating enzyme.

6. The method of claim 5, wherein the E3 ligase is Itch.

7. The method of claim 5, wherein the SUMO-conjugating enzyme is Ubc9.

8. A method for treating a cyclin D1-overexpressing cancer, comprising administering to a subject in need of such treatment a composition comprising a therapeutically effective amount of an agent that increases sumoylation of cyclin D1.

9. The method of claim 8, wherein the cancer is selected from the group consisting of breast cancer, lung cancer, prostate cancer, and bladder cancer.

10. The method of claim 8, wherein the agent is arsenic trioxide.

11. The method of claim 1, further comprising identifying the subject for treatment with an agent that increases sumoylation of cyclin D1, the method comprising:
(a) obtaining a biological sample comprising at least one cancer cell expressing cyclin D1 from the subject; and
(b) identifying the subject as being suitable for treatment with the agent based on detecting at least one sumoylation site in cyclin D1, and identifying the subject as being unsuitable for treatment with the agent based on detecting no sumoylation site in cyclin D1.

12. The method of claim 11, wherein the agent is arsenic trioxide.

13. The method of claim 11, wherein the at least one sumoylation site is a lysine reside in an amino acid sequence of cyclin D1.

14. The method of claim 13, wherein the lysine residue is at position 149 in the amino acid sequence of cyclin D1.

15. The method of claim 11, wherein the subject identified as being suitable for treatment is administered a composition comprising a therapeutically effective amount of the agent.

16. A method for treating cancer in a patient, comprising
determining the presence or absence of at least one sumoylation site in cyclin D1 in a cancer cell from the patient; and
administering a composition comprising a therapeutically effective amount of an agent that mediates downregulation of cyclin D1 if the at the least one sumoylation site in cyclin D1 is present.

17. The method of claim 16, wherein the at the least one sumoylation site in cyclin D1 is a lysine reside in an amino acid sequence of cyclin D1.
